(19) United States
(12) Patent Application Publication
(10) Pub. No.: US 2023/0149435 A1
(43) Pub. Date: May 18, 2023

(54) USE OF NMN FOR THE PREVENTION AND/OR TREATMENT OF MUSCLE, LIGAMENT OR TENDON PAIN INDUCED BY PHYSICAL ACTIVITY AND CORRESPONDING COMPOSITIONS

(71) Applicant: NUVAMID SA, EPALINGES (CH)

(72) Inventors: Guillaume BERMOND, MARSEILLE (FR); Laurent GARCON, SAUSSET LES PINS (FR)

(73) Assignee: NUVAMID SA, EPALINGES (CH)

(21) Appl. No.: 17/910,655

(22) PCT Filed: Mar. 12, 2021
(86) PCT No.: PCT/EP2021/056319

§ 371 Date: Sep. 9, 2022

(30) Foreign Application Priority Data
Mar. 12, 2020 (FR) 2002476

(51) Int. Cl. A61K 31/706; A61K 45/06; A61P 29/00

(52) U.S. Cl. CPC A61K 31/706; A61K 45/06; A61P 29/00

(57) ABSTRACT

Nicotinamide mononucleotide (NMN), a pharmaceutically acceptable derivative thereof, or a pharmaceutically acceptable salt thereof, are described for use thereof in the prevention and/or treatment of a muscle, ligament, tendon or their combination, induced by physical activity; as well as compositions that include the same.

### FIELD OF THE INVENTION

The present invention relates to the use of nicotinamide mononucleotide (NMN), a pharmaceutically acceptable derivative thereof, or a pharmaceutically acceptable salt thereof, as well as compositions that comprise the same, for the prevention and/or treatment of a muscle, ligament, or tendon pain or combinations thereof, induced by physical activity, in particular by the practise of a sporting activity.

### TECHNICAL BACKGROUND

Physical activity, and in particular the practise of a sport, serves to maintain good physical condition and improve one's health. The World Health Organisation (WHO) has developed worldwide recommendations in respect of physical activity for health, that vary in duration and intensity according to the age of the subject (available under ISBN number: 9789241599979).

However, practising physical activity in a manner that is too intensive, poorly executed, or with bad equipment can cause muscle, tendon, or ligament pain or a combination of such pains. For example, it is common to experience muscle pain such as body aches after practising sports, or pain in tendons, ligaments, and muscles when practising high-intensity sports, or an injury while practising a sport.

A muscle is a tissue constituted mainly of contractile cells, or muscle fibres. The muscles enable the movements of the body. There are three types of muscles: skeletal striated muscle, smooth muscle, and cardiac muscle. The present invention concerns those muscle pains that affect the skeletal striated muscles and not the smooth or cardiac muscles. A tendon is situated at the end of a muscle. It is a structure that connects the muscle to the bone, and this structure is very inelastic. The tendon is subjected to enormous traction by the muscles during movement. The ligaments are structures that go from one bone to another bone, in order to maintain the cohesion of a joint during movements. Such pains are therefore not caused by an underlying pathological condition such as osteoarthritis, an inflammatory pathology such as arthritis, or inflammation of the cartilages, a tumour, an autoimmune disease, osteopathy, chondropathy, etc. Nor are they caused by a traumatic condition such as a fracture, dislocation or contusions.

Such pains are generally transient, localised and not very intense. The treatment of such pains often involves self-medication and generally includes the administration of analgesics, non-steroidal anti-inflammatory drugs (NSAIDs) or muscle relaxants administered via topical or oral routes, or by injection.

However, the administration of these drugs causes damage to organs such as the stomach, the liver and the kidneys, among others. In addition, their effectiveness declines over time requiring increased doses.

There therefore exists a need to develop new compositions for the treatment and/or prevention of muscle, ligament, or tendon pain or combinations thereof, induced by physical activity, in particular by the practise of a sport, which reduce the drawbacks of the prior art.

### SUMMARY OF THE INVENTION

These objects are achieved by the invention as described below.

The present invention relates to nicotinamide mononucleotide (NMN), a pharmaceutically acceptable derivative thereof, or a pharmaceutically acceptable salt thereof, for use thereof in the prevention and/or treatment of muscle, ligament, or tendon pain or combinations thereof, induced by physical activity.

Advantageously, the pharmaceutically acceptable derivative of NMN may be dihydronicotinamide mononucleotide (NMN-H).

Advantageously, the pharmaceutically acceptable derivative of NMN may be alpha-NMN.

Advantageously, the pharmaceutically acceptable derivative of NMN may be selected from among: a compound having the formula (I):

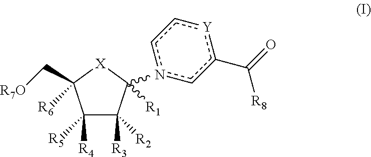

or one of the pharmaceutically acceptable: stereoisomers, salts, hydrates, solvates, or crystals thereof, in which:
X is selected from among O, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄ and R₅ are selected independently of one another, from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from a proteinogenic amino acid;
R₆ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R⁷ is selected from among H, P(O)R₉R₁₀, P(S)R₉R₁₀ and

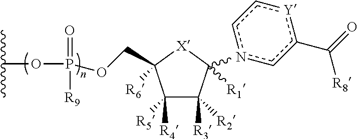

wherein n is an integer selected from 1 or 3; in which
R₉ and R₁₀ are selected independently of one another, from among OH, OR₁₁, NHR₁₃, NR₁₃R₁₄, a (C₁-C₈) alkyl, a (C₂-C₈) alkenyl, a (C₂-C₈)alkynyl, a (C₃-C₁₀) cycloalkyl, a (C₅-C₁₂) aryl, (C₁-C₈)alkyl aryl, (C₁-C₈) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —(CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)ₙC(O)O(C₁-C₁₅)alkyl, and —(CH₂)ₙC(O)O(C₁-C₁₅)alkyl aryl; wherein n is an integer selected from 1 to 8; P(O)(OH)OP(O)(OH)₂; halogen, nitro, cyano, C₁-C₆ alkoxy, C₁-C₆ haloalkoxy, —N(R₁₁ₐ)₂, C₁-C₆ acylamino, —COR_{11b}, —O COR_{11b}; NHSO₂(C₁-C₆ alkyl), —SO₂N(R₁₁ₐ)₂ SO₂; wherein each of R₁₁ₐ is independently selected from H and a (C₁-C₆) alkyl, and Ru_{11b} is independently selected from OH, C₁-C₆ alkoxy, NH₂, NH(C₁-C₆ alkyl) or N(C₁-C₆ alkyl)₂;
R₁₂ is selected from among H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the said aryl or heteroaryl groups are optionally substituted with one or two groups selected from among halogen, trifluoromethyl, C₁-C₆ alkyl, C₁-C₆ alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl, —(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among hydroxyl, (C₁-C₁₀) alkyl, (C₁-C₆) alkoxy, a halogen, a nitro, and a cyano; or
R₉ and R₁₀ form, together with the phosphorus atoms to which they are attached, a 6-membered ring in which —R₉—R₁₀— represents —CH₂—CH₂—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or

R₉ and R₁₀ form, together with the phosphorus atoms to which they are attached, a 6-membered ring in which —R₉—R₁₀— represents —O—CH₂—CH₂—CHR—O—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano;
R₈ is selected from among H, OR, NHR₁₃, NR₁₃R₁₄, NH—NHR₁₃, SH, CN, N₃, and halogen; wherein R₁₃ and R₁₄ are selected independently of one another, from among H, (C₁-C₈) alkyl, (C₁-C₈) alkyl aryl, and —CR_{B}R_{C}—C(O)—OR_{D} in which R_{B} and R_{C} are independently a hydrogen atom, a (C₁-C₆) alkyl, a (C₁-C₆) alkoxy, benzyl, indolyl, or imidazolyl; where the (C₁-C₆) alkyl and the (C₁-C₆) alkoxy may be optionally and independently of one another substituted by one or more of the halogen, amino, amido, guanidyl, hydroxyl, thiol, or carboxyl groups, and the benzyl group is optionally substituted by one or more halogen or hydroxyl groups; or R_{B} and R_{C} form, together with the carbon atom to which they are attached, a C₃-C₆ cycloalkyl group optionally substituted by one or more halogens, amino, amido, guanidyl, hydroxyl, thiol, and carboxyl; and R_{D} is a hydrogen, a (C₁-C₆) alkyl, a (C₂-C₆) alkenyl, a (C₂-C₆) alkynyl, or a (C₃-C₆) cycloalkyl;

Y is selected from among CH, CH₂, C(CH₃)₂ and CCH₃;

[symbol] represents a single or a double bond along Y; and

[symbol] represents the alpha or beta anomer depending on the position of R₁;

or

a compound having the formula (Ia):

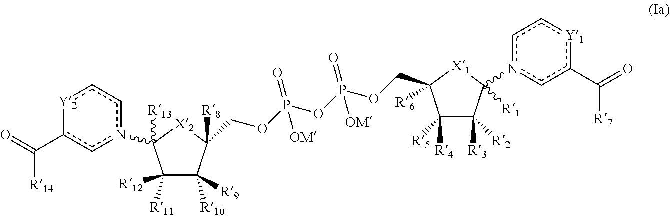

or one of the: stereoisomers, salts, hydrates, solvates, or crystals thereof, in which:

X′₁ and X′₂ are independently selected from among O, CH₂, S, Se, CHF, CF₂, and C═CH₂;

R′₁ and R′13 are independently selected from among H, azido, cyano, a C1-C8 alkyl, a C1-C8 thio-alkyl, a C1-C8 heteroalkyl, and OR, wherein R is selected from H and a C1-C8 alkyl;

R′₂, R′₃, R′₄, R′₅, R′₉, R′₁₀, R′₁₁, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR;

wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH2 group; wherein R_{AA} is a side chain selected from a proteinogenic amino acid;

R′₆ and R′₈ are independently selected from among H, an azido, a cyano, a C₁-C₈ alkyl and OR, wherein R is selected from H and a C₁-C₈ alkyl;

R′₇ and R′₁₄ are independently selected from among H, OR, NHR, NRR′, NH—NHR, SH, CN, N₃ and a halogen; wherein R and R′ are independently selected from H and a (C₁-C₈) alkyl aryl;

Y′₁ and Y′₂ are independently selected from among CH, CH₂, C(CH₃)₂, or CCH₃;

M′ is selected from H or a suitable counter ion;

[symbol] represents a single or double bond depending on Y′₁ and Y′₂; and [symbol] represents an alpha or beta anomer depending on the position of R′₁ and R′₁₃;

and combinations thereof.

In a first preferred embodiment, the pharmaceutically acceptable derivative is the compound having the formula (I).

In one variant of the first embodiment, X represents an oxygen.

In one variant of the first embodiment, R₁ and R₆ each independently of one another represent a hydrogen.

In one variant of the first embodiment, R₂, R₃, R₄ and R₅ each independently of one another represent a hydrogen or an OH.

In one variant of the first embodiment, Y represents a CH.

In one variant of the first embodiment, Y represents a CH₂.

In one variant of the first embodiment, R₇ represents a hydrogen.

In one variant of the first embodiment, R₇ represents P(O)(OH)₂.

In one variant of the first embodiment,

X represents an oxygen; and/or

R₁ and R₆ each independently represent hydrogen; and/or

R₂, R₃, R₄ and R₅ each independently represent hydrogen, or R₂, R₃, R₄ and R₅ independently represent OH; and/or

Y represents a CH or a CH₂; and/or

R₇ represents P(O)R₉R₁₀, wherein R₉ and R₁₀ are independently selected from among OH, OR₁₁, NHR₁₃, NR₁₃R₁₄, C₁-C₈ alkyl, C₂-C₈ alkenyl, C₂-C₈ alkynyl, C₃-C₁₀ cycloalkyl, C₅-C₁₂ aryl, C₁-C₈ aryl alkyl, C₁-C₈ alkyl aryl, C₁-C₈ heteroalkyl, C₁-C₈ heterocycloalkyl, heteroaryl, and NHCR_{A}R_{A′}C(O)R₁₂.

In one particularly preferred variant of the first embodiment, the compound of the invention is selected from among the compounds having the formula IB to IJ:

TABLE 1

| Compounds | |
|---|---|
| No (Anomers) | Structure |
| I-B (alpha) | 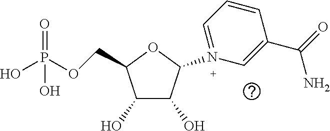 |
| I-C (beta) | 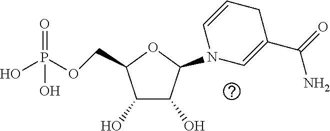 [SMILES: NC(=O)C1=CN([C@@H]2O[C@H](COP(=O)(O)O)[C@@H](O)[C@H]2O)C=CC1] |
| I-D (alpha) | 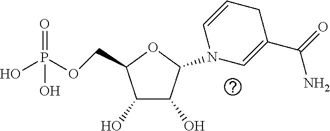 [SMILES: NC(=O)C1=CN([C@H]2O[C@H](COP(=O)(O)O)[C@@H](O)[C@H]2O)C=CC1] |
| I-E (beta) | 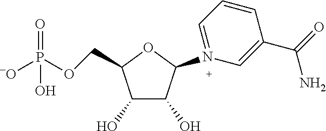 |
| I-F (alpha) | 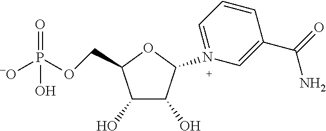 |
| I-G (beta) | 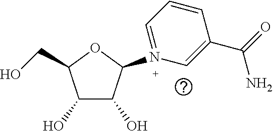 |
| I-H (alpha) | 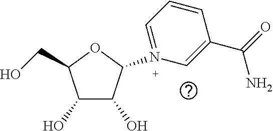 |
| I-I (beta) | 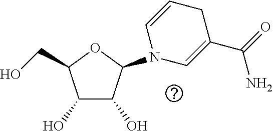 [SMILES: NC(=O)C1=CN([C@@H]2O[C@H](CO)[C@@H](O)[C@H]2O)C=CC1] |
| I-J (alpha) | 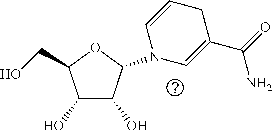 [SMILES: NC(=O)C1=CN([C@H]2O[C@H](CO)[C@@H](O)[C@H]2O)C=CC1] |

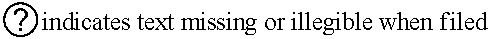

Advantageously, the pharmaceutically acceptable derivative of NMN may be alpha-NMN (compounds I-F).

Advantageously, the pharmaceutically acceptable derivative of NMN may be dihydronicotinamide mononucleotide (NMN-H) (compounds I-C or I-D).

In a preferred second embodiment, the pharmaceutically acceptable derivative is the compound having the formula (Ia).

In one variant of the second embodiment, X′1 and X′2 each independently represent an oxygen.

In one variant of the second embodiment, R′7 and R′14 each independently represent an NH₂.

In one variant of the second embodiment, R′1 and/or R′13 each independently represent a hydrogen.

In one variant of the second embodiment, R′6 and/or R′8 each independently represent a hydrogen.

In one variant of the second embodiment, R′2, R′3, R′4, R′5, R′9, R′10, R′11, and R′12 each independently represent a hydrogen.

In one variant of the second embodiment, R′2, R′3, R′4, R′5, R′9, R′10, R′11, and R′12 each independently represent an OH.

In one variant of the second embodiment, Y′1 and Y′2 each independently represent a CH.

In one variant of the second embodiment, Y′1 and Y′2 each independently represent a CH2.

In one variant of the second embodiment, the compound according to the invention is selected from among the compounds having the formula Ia-A to Ia-I:

TABLE 2

| Compounds | |
|---|---|
| (Anomers) | Structure |
| Ia-A (beta, beta) | 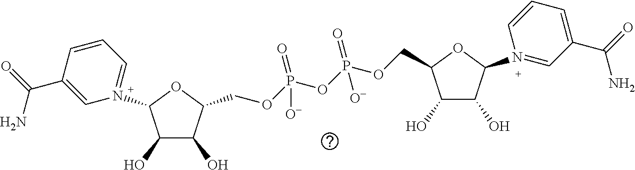 |
| Ia-B (beta, alpha) | 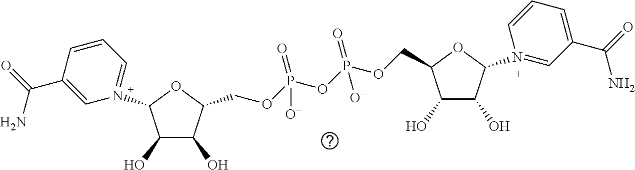 |
| Ia-C (alpha, alpha) | 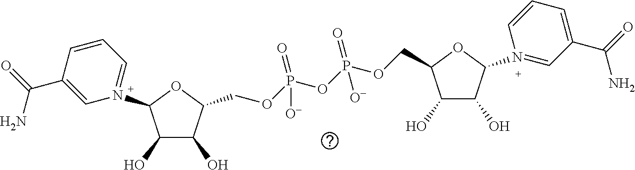 |
| Ia-D (beta, beta) | 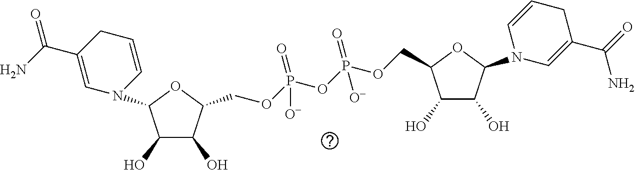 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| Ia-E (beta, alpha) | 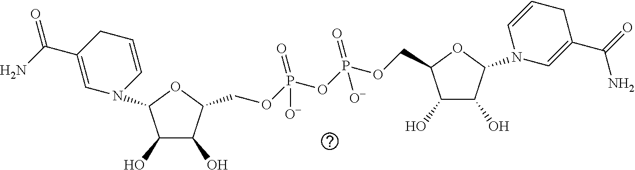 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-F (alpha, alpha) | 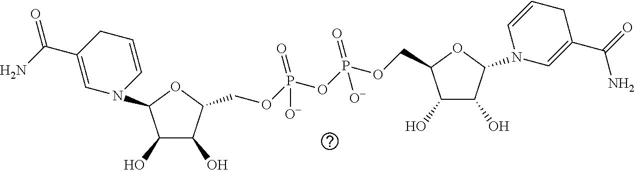 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-G (beta, beta) | 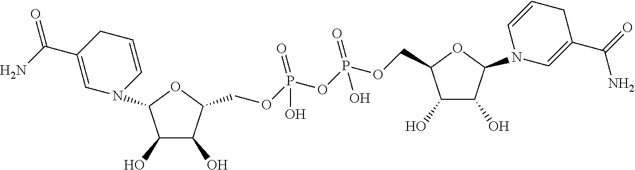 [SMILES: NC(=O)C1=CN([C@@H]2O[C@H](COP(=O)(O)OP(=O)(O)OC[C@H]3O[C@@H](N4C=CCC(C(N)=O)=C4)[C@H](O)[C@@H]3O)[C@@H](O)[C@H]2O)C=CC1] |
| Ia-H (beta, alpha) | 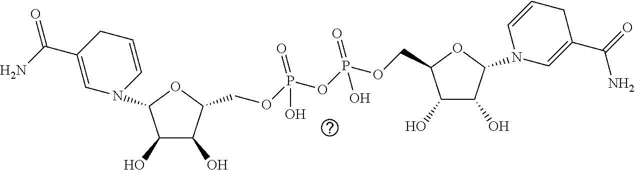 [SMILES: NC(=O)C1=CN([C@@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |
| Ia-I (alpha, alpha) | 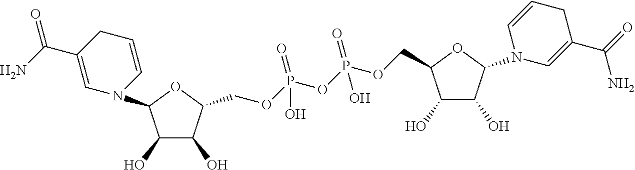 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

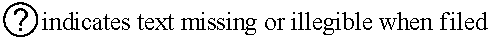

Advantageously, NMN, a pharmaceutically acceptable derivative thereof, or a pharmaceutically acceptable salt thereof, may be used in the treatment and/or prevention of a muscle, ligament, or tendon pain or combinations thereof, induced by physical activity, in mammals and preferably in humans.

Preferably, the NMN, a pharmaceutically acceptable derivative thereof, or a pharmaceutically acceptable salt thereof, is intended to be administered via the topical route.

Advantageously, the pain is not due to one of the pathologies selected from the following: a tumour, arthritis, gout, osteoarthritis, a joint deformity, a connective tissue disease, dorsopathy, a neurodegenerative disease, neuropathy, a genetic disease, an autoimmune disease, myopathy, osteopathy, osteoporosis, chondropathy, vasculopathy, a viral infection, a fungal infection, a bacterial infection, a parasite, adverse side effects of a medication, a surgical procedure, a medical examination, calcification, trauma (unless induced by a physical activity), a malformation or combinations thereof.

Advantageously, the muscle pain may be selected from among: soreness, contracture, cramping, elongation (pulled/strained muscles), muscle contusion, muscle tearing, partial or complete rupture of muscle fibres, or combinations thereof.

Advantageously, the ligament pain may be a sprain, a partial or complete tearing of the ligament or combinations thereof.

Advantageously, the tendon pain may be tendonitis, tenosynovitis, bursitis, or combinations thereof.

Preferably, the pain is a muscle pain.

In one preferred embodiment, the physical activity is the practising of a sport.

Advantageously, the NMN, a pharmaceutically acceptable derivative thereof, or a pharmaceutically acceptable salt thereof, is intended to be administered between 1 and 10 times per day, preferably between 1 and 5 times per day, more preferably between 1 and 3 times a day.

In a preferred embodiment, the NMN, a pharmaceutically acceptable derivative thereof, or a pharmaceutically acceptable salt thereof, is intended to be administered twice a day.

Advantageously, the NMN, a pharmaceutically acceptable derivative thereof, or a pharmaceutically acceptable salt thereof, may be used in combination with at least one other therapeutic agent.

Advantageously, the at least one therapeutic agent may be an analgesic, a non-steroidal anti-inflammatory drug, cortisone, a cortisone derivative, a muscle relaxant, arnica, salicylic acid, aescin, capsaicin, zucapsaicin, tolazoline, dimethyl sulfoxide, idrocilamide, or combinations thereof.

Advantageously, the analgesic may be selected from among paracetamol, nefopam, ketanin, tetrahydrocannabinol, cannabinoids, aspirin, methyl salicylate, diflunisal, salicylamide, codeine, alfentanil, carfentanil, dihydrocodeine, codeinone, tramadol, morphine, morphinone, buprenorphine, fentanyl, acetyl fentanyl, remifentanil, sufentanil, heroin, hydromorphone, nalbuphine, oxycodone, hydroxycodone, oxymorphone, laudanum, methadone, pethidine, dextropropoxyphene, endorphin, tapentadol, thebaine, vicodin, and combinations thereof.

Advantageously, the non-steroidal anti-inflammatory drug may be selected from among ibuprofen, ketoprofen, naproxen, ketorolac, alminoprofen, aceclofenac, mefenamic acid, niflumic acid, tiaprofenic acid, celecoxib, rofecoxib, valdecoxib, parecoxib, dexketoprofen, diclofenac, etodolac, etoricoxib, fenoprofen, flurbiprofen, indomethacin, meloxicam, nabumetone, piroxicam, sulindac, tenoxicam, nimesulide, and combinations thereof.

Advantageously, the cortisone derivative may be selected from among betamethasone, ciprofloxacin, cortivazol, dexamethasone, fludrocortisone, methylprednisolone, prednisolone, triamcinolone, and combinations thereof.

Advantageously, the muscle relaxant may be selected from among locally acting muscle relaxants, centrally acting muscle relaxants, carbamic acid esters and derivatives thereof, as well as combinations thereof.

Advantageously, the muscle relaxant may be selected from among baclofen, quinine, mephenesin, tizanidine, tetrazepam, thiocolchicoside, acetyl hexapeptide-8, μ-conotoxin CnIIIc (mu-conotoxin CnIIIc), dipeptide diaminobutyroyl benzylamide diacetate as well as locally used botulinum toxin, and combinations thereof.

Preferably, the muscle relaxant is a locally acting muscle relaxant.

Advantageously, the carbamic acid ester may be methocarbamol.

Advantageously, nicotinamide mononucleotide (NMN), a pharmaceutically acceptable derivative thereof, or a pharmaceutically acceptable salt thereof, makes it possible to reduce the muscle, ligament, or tendon stiffness or combinations thereof.

Advantageously, nicotinamide mononucleotide (NMN), a pharmaceutically acceptable derivative thereof, or a pharmaceutically acceptable salt thereof, makes it possible to improve the muscle, ligament, or tendon function or combinations thereof.

The present invention also relates to a composition comprising nicotinamide mononucleotide (NMN), a pharmaceutically acceptable derivative thereof, or a pharmaceutically acceptable salt thereof, and at least one pharmaceutically acceptable excipient, for use thereof in the prevention and/or treatment of a muscle pain, ligament pain, tendon pain or combinations thereof, resulting from physical activity. Preferably, the composition according to the invention is intended to be administered via the topical route.

Advantageously, the composition according to the invention may be in the form of a gel, a solution, a water-in-oil emulsion, an oil-in-water emulsion, an oil, a cream, an ointment/salve, or a liniment.

In one preferred embodiment, the composition according to the invention is in the form of a water-in-oil emulsion or an oil-in-water emulsion; on a more preferred basis, an oil-in-water emulsion.

In a more preferred embodiment, the composition according to the invention is in the form of a hydrophilic or lipophilic gel, in a more preferred manner in the form of a hydrophilic gel.

Advantageously, the composition according to the invention may be a pharmaceutical composition.

Advantageously, the composition according to the invention may comprise NMN, a pharmaceutically acceptable salt thereof, or a pharmaceutically acceptable derivative thereof, in an amount comprised between 0.05% and 15% by weight, preferably between 1 and 10% by weight, on a more preferred basis between 3 and 5% by weight relative to the total weight of the composition.

Advantageously, the composition according to the invention, may be administered between 1 and 10 times per day, preferably between 1 and 5 times per day, on a more preferred basis between 1 and 3 times per day.

In one preferred embodiment, the composition according to the invention, may be administered twice a day.

Advantageously, the composition according to the invention may also comprise at least one additional therapeutic agent as defined above for use thereof in the prevention and/or treatment of a muscle pain, ligament pain or tendon pain or combinations thereof, induced by physical activity as described above.

### DEFINITIONS

In the present invention, the following terms have the following meanings.

Unless otherwise indicated, the nomenclature of substituents which are not explicitly defined in the present invention is obtained by naming the terminal portion of the functional group followed by the adjacent functional group towards the point of attachment.

“Alkyl” by itself or as part of another substituent refers to a hydrocarbyl radical having the formula CnH2n+1 in which n is a number greater than or equal to 1. In general, the alkyl groups of this invention include from 1 to 12 carbon atoms, preferably from 1 to 8 carbon atoms, more preferably from 1 to 6 carbon atoms, even more preferably from 1 to 2 carbon atoms. The alkyl groups may be linear or branched and may be substituted as indicated in the present invention. The alkyls that are suitable for the purposes of implementation of the invention may be selected from among methyl, ethyl, n-propyl, i-propyl, n-butyl, i-butyl, s-butyl and t-butyl; pentyl and its isomers such as n-pentyl and iso-pentyl; and hexyl and its isomers such as n-hexyl and iso-hexyl; heptyl and its isomers (for example n-heptyl, iso-heptyl); octyl and its isomers (for example n-octyl, iso-octyl); nonyl and its isomers (for example n-nonyl, iso-nonyl); decyl and its isomers (for example n-decyl, iso-decyl); undecyl and its isomers; dodecyl and its isomers. Preferably, the alkyl groups may be selected from among methyl, ethyl, n-propyl, i-propyl, n-butyl, i-butyl, s-butyl, t-butyl, n-pentyl, n-hexyl, n-heptyl, n-octyl, n-nonyl, and n-decyl. The saturated and branched alkyl groups may be selected, without limitation, from among isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, and 3,3-diethylhexyl. The preferred alkyl groups are the following: methyl, ethyl, n-propyl, i-propyl, n-butyl, butyl, s-butyl and t-butyl. Cx-Cy-alkyls refer to alkyl groups that contain from x to y carbon atoms.

When the suffix “ene” (“alkylene”) is used in conjunction with an alkyl group, it indicates that the alkyl group as defined herein has two single bonds as points of attachment to other groups. The term “alkylene” includes methylene, ethylene, methylmethylene, propylene, ethylethylene, and 1,2-dimethylethylene.

The term “alkenyl” as used herein refers to an unsaturated hydrocarbyl group, which may be linear or branched, that comprises one or more carbon-carbon double bonds. The alkenyl groups that are suitable comprise between 2 and 12 carbon atoms, preferably between 2 and 8 carbon atoms, and even more preferably between 2 and 6 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and other similar groups.

The term “alkynyl”, as used herein, refers to a class of monovalent unsaturated hydrocarbyl groups, in which the unsaturation results from the presence of one or more carbon-carbon triple bond(s). The alkynyl groups generally, and preferably, have the same number of carbon atoms as described here above for the alkenyl groups. Without limitation, some examples of alkynyl groups include ethynyl, 2-propynyl, 2-butynyl, 3-butynyl, 2-pentynyl and its isomers, 2-hexynyl and its isomers, etc.

“Alkoxy” refers to an alkyl group as defined here above, which is attached to another moiety by means of an oxygen atom. Examples of alkoxy groups include the groups: methoxy, isopropoxy, ethoxy, tert-butoxy, and the like. The alkoxy groups may be optionally substituted by one or more substituent(s). The alkoxy groups included in the compounds of this invention may be optionally substituted with a solubilising group.

“Aryl”, as used herein, refers to a polyunsaturated aromatic hydrocarbyl group having a single ring (for example phenyl) or multiple aromatic rings that are fused together (for example naphthyl) or covalently bonded, which generally contains 5 to 18 atoms, preferably 5 to 12, on a more preferred basis 6 to 10, with at least one of the said rings being aromatic. The aromatic ring may optionally include one or two additional rings (cycloalkyl, heterocyclyl, or heteroaryl) fused thereto. The aryl is also intended to include partially hydrogenated derivatives of the carbocyclic systems listed herein. Examples of aryl include phenyl, biphenylyl, biphenylenyl, 5- or 6-tetralinyl; naphthalene-1-or -2-yl; 4-, 5-, 6 or 7-indenyl; 1-, 2-, 3-, 4-, or 5-acenaphthylenyl; 3-, 4-, or 5-acenaphthenyl; 1-, or 2-pentalenyl; 4-, or 5-indanyl; 5-, 6-, 7-, or 8-tetrahydronaphthyl; 1,2,3,4-tetrahydronaphthyl; 1,4-dihydronaphthyl; 1-, 2-, 3-, 4-, or 5-pyrenyl.

When at least one carbon atom in an aryl group is replaced by a heteroatom, the resulting ring is referred to herein as a “heteroaryl” ring.

“Alkylaryl” refers to an aryl group substituted by an alkyl group.

“Amino acid” refers to an alpha-amino carboxylic acid, that is to say, a molecule comprising a carboxylic acid functional group and an amino functional group in the alpha position of the carboxylic acid group, for example a proteinogenic amino acid or a non-proteinogenic amino acid.

“Proteinogenic amino acid” refers to an amino acid that is incorporated into the proteins during the translation of the messenger RNA by the ribosomes in living organisms, that is to say, Alanine (ALA), Arginine (ARG), Asparagine (ASN), Aspartate (ASP), Cysteine (CYS), Glutamate (glutamic acid) (GLU), Glutamine (GLN), Glycine (GLY), Histidine (HIS), Isoleucine (ILE), Leucine (LEU), Lysine (LYS), Methionine (MET), Phenylalanine (PHE), Proline (PRO), Pyrrolysine (PYL), Selenocysteine (SEL), Serine (SER), Threonine (THR), Tryptophan (TRP), Tyrosine (TYR), or Va line (VAL).

“Non-proteinogenic amino acid” as used herein refers to an amino acid that is not naturally encoded or found in the genetic code of a living organism. Without limitation, some examples of non-proteinogenic amino acid are: ornithine, citrulline, argininosuccinate, homoserine, homocysteine, cysteine-sulfinic acid, 2-aminomuconic acid, δ-aminolevulinic acid, β-alanine, cystathionine, γ-aminobutyrate, dihydroxyphenylalanine (DOPA), 5-hydroxytryptophan, D-serine, ibotenic acid, α-aminobutyrate, 2-aminoisobutyrate, D-leucine, D-valine, D-alanine, and D-glutamate.

The term “cycloalkyl” as used herein refers to a cyclic alkyl group, that is to say, a monovalent, saturated or unsaturated hydrocarbyl group, having 1 or 2 ring structures. The term “cycloalkyl” includes monocyclic or bicyclic hydrocarbyl groups. The cycloalkyl groups may comprise 3 or more carbon atom(s) in the ring and generally, according to the present invention, comprise from 3 to 10, more preferably from 3 to 8 carbon atoms, and even more preferably from 3 to 6 carbon atoms. Examples of cycloalkyl groups include, but are not limited to, cyclopropyl, cyclobutyl, cyclopentyl, cyclohexyl, with cyclopropyl being particularly preferred.

The term “pharmaceutically acceptable excipient” refers to an inert carrier or support substance used as a solvent or diluent within which the active ingredient is formulated and/or administered, and which does not produce an adverse, allergic or other reaction when it is administered to an animal, preferably to a human. This includes all solvents, dispersing media, coatings, antibacterial and antifungal agents, isotonic agents, absorption retardants, and other similar ingredients. For human administration, the preparations must meet specific standards of sterility, general safety and purity, as required by the regulatory authorities, such as for example the Food and Drug Administration (FDA) in the United States of America, or the European Medicines Agency (EMA). Within the meaning of the invention, “pharmaceutically acceptable excipient” includes all pharmaceutically acceptable excipients as well as all pharmaceutically acceptable carriers, diluents and/or adjuvants.

“Halogen” or “halo” refers to fluoro, chloro, bromo or iodo. The preferred halo groups are fluoro and chloro.

“Haloalkyl” alone or in combination, refers to an alkyl radical having the meaning as defined here above, in which one or more hydrogen atom(s) are replaced by a halogen as defined here above. By way of examples of such haloalkyl radicals, the following may be cited: chloromethyl, 1-bromoethyl, fluoromethyl, difluoromethyl, trifluoromethyl, 1,1,1-trifluoroethyl, and similar radicals. ‘Cx-Cy-haloalkyl’ and ‘Cx-Cy-alkyl’ refer to alkyl groups that contain from x to y carbon atoms. The preferred haloalkyl groups are difluoromethyl and trifluoromethyl.

“Heteroalkyl” refers to an alkyl group as defined here above, in which one or more carbon atom(s) are replaced by a heteroatom selected from among oxygen, nitrogen and sulfur atoms. In the heteroalkyl groups, the heteroatoms are bonded along the alkyl chain only to carbon atoms, that is to say, each heteroatom is separated from every other heteroatom by at least one carbon atom. However, the nitrogen and sulfur heteroatoms may optionally be oxidised and the nitrogen heteroatoms may optionally be quaternised. A heteroalkyl is bonded to another group or molecule only by means of a carbon atom, that is to say, the bonding atom is not selected from the heteroatoms included in the heteroalkyl group.

The term “heteroaryl” as used herein, alone or as part of another group, refers to, but is not limited to, aromatic rings of 5 to 12 carbon atoms or ring systems containing 1 or 2 rings that are fused or covalently bonded, and generally containing 5 or 6 atoms, with at least one of the said rings being aromatic; in which one or more carbon atom(s) in one or more of these rings are replaced by oxygen, nitrogen and/or sulfur atoms, it being possible for the nitrogen and sulfur heteroatoms to optionally be oxidised and for the nitrogen heteroatoms to optionally be quaternised. These rings may be fused to an aryl, cycloalkyl, heteroaryl or heterocyclyl ring. Without limitation, some examples of such heteroaryls include: furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, dioxinyl, thiazinyl, triazinyl, imidazo [2,1-b] [1,3] thiazolyl, thieno [3,2-b] furanyl, thieno [3,2-b] thiophenyl, thieno [2,3-d] [1,3] thiazolyl, thieno [2,3-d] imidazolyl, tetrazolo [1,5-a] pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo [I,2-a] pyridinyl, 6-oxo-pyridazin-1(6H)-yl, 2-oxopyridin-1(2H)-yl, 6-oxo -pyridazin-1(6H)-yl, 2-oxopyridin-1(2H)-yl, 1,3-benzodioxolyl, quinolinyl, isoquinolinyl, cinnolinyl, quinazolinyl, quinoxalinyl.

When at least one carbon atom in a cycloalkyl group is replaced by a heteroatom, the resulting ring is referred to herein as “heterocycloalkyl” or “heterocyclyl”.

The terms “heterocyclyl”, “heterocycloalkyl”, or “heterocyclo”, as used herein by themselves or as part of another group, refer to non-aromatic cyclic groups, either fully saturated or partially unsaturated (for example, 3 to 7 membered monocyclic, 7 to 11 membered bicyclic groups or containing a total of 3 to 10 ring atoms), which have at least one heteroatom in at least one ring containing a carbon atom. Each ring of the heterocyclic group containing a heteroatom may have 1, 2, 3, or 4 heteroatoms selected from among nitrogen, oxygen and/or sulfur atoms, where the nitrogen and sulfur heteroatoms may optionally be oxidised, and the nitrogen heteroatoms may optionally be quaternised. Any whichever of the carbon atoms of the heterocyclic group may be substituted by an oxo (for example piperidone, pyrrolidinone). The heterocyclic group may be attached to any heteroatom or carbon atom in the ring or ring system, where the valence so permits. The rings of multi-ring heterocycles may be fused, bridged and/or connected by one or more spiro atoms. Exemplary heterocyclic groups include, but are not limited to, the following groups: oxetanyl, piperidinyl, azetidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholine-4-yl, thiomorpholine-4-ylsulfoxide, thiomorpholine-4-ylsulfone, 1,3-dioxolanyl, 1,4-oxathianyl, 1H-pyrrolizinyl, tetrahydro-1,1-dioxothiophenyl, N-formylpiperazinyl, and morpholine-4-yl.

The term “precursor” as used herein also refers to pharmacologically acceptable derivatives of compounds having the formula (I) or (Ia) such as esters, of which the in vivo biotransformation product is the active drug. Precursors are characterised by increased bioavailability and are readily metabolised into active compounds in vivo. The precursors that are appropriate for the purposes of the invention include in particular carboxylic esters, in particular alkyl esters, aryl esters, acyloxyalkyl esters, and the carboxylic esters of dioxolene; ascorbic acid esters.

The term “pharmaceutically acceptable” refers to the state of being approved, or with the likelihood of being potentially approved by a regulatory body or listed in a recognised pharmacopoeia for use in animals, and more preferably in humans. It may pertain to a substance that is not biologically or otherwise undesirable; that is to say, the substance may be administered to an individual without causing adverse biological effects or deleterious interactions with one of the components of the composition within which it is contained. Preferably, a “pharmaceutically acceptable” salt or excipient refers to any salt or any excipient that is authorised by the European Pharmacopoeia (denoted as “Ph. Eur.”) and the American Pharmacopoeia (generally referred to as “United States Pharmacopeia (USP)”).

The term “active ingredient” or “therapeutic agent” refers to a molecule or a substance which when administered to a subject slows down or stops the progression, aggravation or deterioration of one or more symptom(s) of a disease or a condition; relieves the symptoms of a disease or a condition; cures a disease or a condition. According to one of these embodiments, the therapeutic ingredient is a small molecule, which is natural or synthetic. According to another embodiment, the therapeutic ingredient is a biological molecule such as, for example, an oligonucleotide, a small interfering RNA (siRNA), a microRNA (miRNA), a DNA fragment, an aptamer, an antibody and the like. “Pharmaceutically acceptable salts” include the acid addition salts and base addition salts of these said salts. Suitable acid addition salts are formed from acids that form non-toxic salts. Examples that may be cited include: acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/ bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/ hydrogen phosphate/dihydrogen phosphate, pyroglutamate, saccharate, stearate, succinate, tannate, tartrate, tosylate, trifluoroacetate, and salts of xinofoate. Suitable basic salts are formed from bases which form non-toxic salts. By way of examples, mention may be made of the salts of: aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine, and zinc. Hemisalts of acids and bases may also be formed, for example, hemisulfates and salts of chemical calcium. The preferred pharmaceutically acceptable salts are hydrochloride/chloride, bromide/ hydrobromide, bisulfate/sulfate, nitrate, citrate and acetate.

Pharmaceutically acceptable salts may be prepared by one or more of the following methods:
by reacting the compound with the desired acid;
by reacting the compound with the desired base;
by removing an acid or base labile protecting group under basic or acidic conditions from a suitable precursor of the compound, or by ring opening of a suitable cyclic precursor, for example a lactone or a lactam, using the desired acid; or
by converting one salt of the compound into another by reacting the initial salt with an appropriate acid or by means of an appropriate ion exchange column.

All of these reactions are generally carried out in solution. The salt can precipitate out of the solution and may be collected by filtration or may be recovered by evaporation of the solvent. The degree of ionisation of the salt may vary from completely ionised to almost non-ionised.

The term “Solvate” is used herein to describe a molecular complex that comprises the compound of the invention and one or more pharmaceutically acceptable solvent molecules, for example, ethanol.

The term “substituent” or “substituted” indicates that a hydrogen radical on a compound or a group is replaced by any desired group which is substantially stable under the reaction conditions in an unprotected form or when it is protected by a protecting group. Examples of preferred substituents include, but are not limited to: a halogen (chloro, iodo, bromo, or fluoro); an alkyl; an alkenyl; an alkynyl, as described here above; a hydroxy; an alkoxy; a nitro; a thiol; a thioether; an imine; a cyano; an amido; a phosphonato; a phosphine; a carboxyl; a thiocarbonyl; a sulfonyl; a sulfonamide; a ketone; an aldehyde; an ester; an oxygen (—O); a haloalkyl (for example, trifluoromethyl); a cycloalkyl, which may be condensed-ring or non-condensed-ring monocyclic or polycyclic (for example, cyclopropyl, cyclobutyl, cyclopentyl or cyclohexyl); or a heterocycloalkyl, which may be condensed-ring or non-condensed-ring monocyclic or polycyclic (for example, pyrrolidinyl, piperidinyl, piperazinyl, morpholinyl, or thiazinyl); fused or unfused monocyclic or polycyclic, aryl or heteroaryl (for example, aryl, heteroaryl, pyrrolidinyl, piperidinyl, piperazinyl, morpholinyl, or thiazinyl); fused or unfused monocyclic or polycyclic (for example, aryl, heteroaryl, pyrrolidinyl, piperidinyl, piperazinyl, morpholinyl, or thiazinyl), phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazidinyl, pyridaziminyl, pyridaziminyl, benzimidazolyl, benzothiophenyl, or benzofuranyl); amino (primary, secondary or tertiary); CO₂CH₃; CONH₂; OCH₂CONH₂; NH₂; SO₂NH₂ ; OCHF₂; FC₃; OCF₃; moreover these groups may also be optionally substituted by a fused ring bridge or structure, for example —OCH₂O—. These substituents may optionally be further substituted by a substituent selected from among these groups. In certain representations, the term “substituent” or the adjective “substituted” refers to a substituent selected from the group constituted of: an alkyl, an alkenyl, an alkynyl, a cycloalkyl, a cycloalkenyl, a heterocycloalkyl, an aryl, a heteroaryl, an arylalkyl, a heteroarylalkyl, a haloalkyl, —C(O)NR₁₁R₁₂, —NR₁₃C(O)R₁₄, a halo, —OR₁₃, cyano, nitro, a haloalkoxy, —C(O)R₁₃, NR₁₁R₁₂, SR₁₃, —C(O)OR′₁₃, —OC(O)R₁₃, —NR₁₃C(O)NR₁₁R₁₂, —OC(O)NR₁₁R₁₂, —NR₁₃C(O)OR₁₄, —S(O)rR13, —NR₁₃S(O)rR₁₄, —OS(O)rR₁₄, S(O)rNR₁₁R₁₂, —O, —S, and —NR₁₃, where r is 1 or 2; R₁₁ and R₁₂, for each occurrence, are independently H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl; or R₁₁ and R₁₂ taken together with the nitrogen to which they are attached are an optionally substituted heterocycloalkyl, or an optionally substituted heteroaryl; and R₁₃ and R₁₄ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl. In certain variants, the term “substituent” or the adjective “substituted” refers to a solubilising group.

The term “administration”, or a variant of this term (for example, “administer”), refers to providing of the active ingredient, whether alone or as part of a pharmaceutically acceptable composition, to the patient who is to receive the same in the context of treatment or prevention of a condition, a symptom, or a disease.

The terms “treating”, “curing”, and “treatment”, as used herein, are meant to include the relieving, alleviation, or ablation of a condition, or a disease and/or the symptoms associated therewith.

The terms “prevent”, “impede” and “prevention”, as used in the present invention, refer to a method that serves the purpose of: delaying, or impeding or preventing the onset of a condition, or a disease and/or the symptoms associated therewith; preventing a patient from contracting a condition or a disease; or reducing the risk of a patient's contracting a given disease or a condition.

The bonds of an asymmetric carbon may be represented herein using a solid triangle ( [symbol] ) a dotted triangle ( [symbol] ) or a zigzag line ( [symbol] ).

### DETAILED DESCRIPTION OF THE INVENTION

The object of the present invention relates to nicotinamide mononucleotide (NMN), a pharmaceutically acceptable derivative thereof, or a pharmaceutically acceptable salt thereof, for use thereof via topical administration in the prevention and/or treatment of muscle, ligament or tendon pain or combinations thereof, induced by physical activity.

Nicotinamide adenine dinucleotide (NAD) is a coenzyme present in all living cells. NAD exists in the cell either in its oxidised form NAD+, or in its reduced form NADH. The role of NAD is that of an electron carrier that is involved in the oxidation-reduction reactions of metabolism. NAD is moreover also involved in a number of cellular processes such as adenosine diphosphate (ADP) ribosylation in the context of post-translational modifications of proteins.

NAD may be synthesised de novo by the cell from amino acids such as tryptophan or aspartate. However, such synthesis is marginal because the main pathway for NAD synthesis is the salvage pathway, by means of which the cell, and primarily the cell nucleus, recycles compounds in order to reform NAD from precursors. The precursors of NAD include niacin, nicotinamide riboside, nicotinamide mononucleotide, and nicotinamide.

NMN is one of the compounds that enable the synthesis of NAD by the salvage pathway and has the formula:

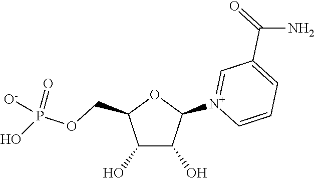 [SMILES: NC(=O)c1ccc[n+]([C@@H]2O[C@H](COP(=O)([O-])O)[C@@H](O)[C@H]2O)c1]

The inventors have in fact demonstrated that the use of NMN, of the pharmaceutically acceptable salts and/or derivatives thereof and of the composition according to the invention makes it possible to relieve muscle pain, ligament pain, tendon pain or combinations thereof, resulting from physical activity, and in particular from the practise of a sport.

The inventors have in fact demonstrated that the use of NMN, of a pharmaceutically acceptable derivative thereof, or a pharmaceutically acceptable salt thereof, as well as compositions that comprise the same, proves to be particularly effective in reducing muscle, ligament, or tendon pain or combinations thereof, in particular muscle pain, induced by physical activity, in particular sports activity.

More precisely, the inventors have demonstrated that the use of NMN, of a pharmaceutically acceptable derivative thereof, or a pharmaceutically acceptable salt thereof, as well as compositions that comprise the same, makes it possible to reduce muscle, ligament, or tendon pain or combinations thereof—preferably muscle pain, in a sufficiently effective manner so as to avoid the need for conventional therapies. The inventors have in fact demonstrated that the subjects who had been administered the NMN or the compositions that comprise the same according to the invention, were able to avoid having to resort to conventional therapies to treat their muscle, ligament or tendon pain, in particular their muscle pain.

According to the WHO, the term “physical activity” is defined as any bodily movement produced by skeletal muscles that requires energy expenditure. Physical activity may be linked to professional activity. Leisure physical activities may include sports as also activities carried out without supervision: for example, walks, bike rides, scooter rides, in parks and green spaces, in the countryside; free-access specialised equipment may be used to perform physical activities (equipped fitness trails, multi-sports grounds, outdoor fitness areas, bicycle paths, hiking circuits, etc). Domestic/household activities pertain to physical activities carried out at home, whether inc or out activites domestiques (ascending and descending stairs, housework—vacuuming, carrying groceries, DIY, gardening). Finally, in one preferred embodiment, the invention's applicability extends to muscle, ligament, tendon pains and combinations thereof resulting from the practise of a sport.

According to the WHO, practising a sport or “doing exercise” corresponds to a subcategory of physical activity that is more deliberate, structured, repetitive, and which aims to improve or maintain one or more aspects of physical condition and fitness. In the context of the present invention, the practise of a sport, sports and sporting activity are referred to without distinction. According to ANSES (Agence Nationale de Sécurité Sanitaire de l'alimentation, de l'environnement et du Travail/French Agency for Food, Environmental and Occupational Health & Safety), sports can also be defined as a physical activity where participants adhere to a common set of rules and where a performance objective is defined (for example: team sports, gymnastics, water gymnastics/aquatic exercise, running, Nordic walking, cycling, cross-country skiing, rowing, swimming).

Global recommendations in respect of physical activity for health have been developed by the WHO taking into account the age of the target population and the intensity of the physical activity. For example, the WHO physical activity guidelines recommend that children and adolescents aged 5 to 17 should get at least 60 minutes of physical activity of moderate-to-vigorous intensity per day, with the knowledge that more than 60 minutes per day of physical activity will provide additional health benefits; and that muscle and bone strengthening activities should each be incorporated at least 3 days per week. Furthermore, WHO physical activity guidelines recommends that adults aged 18-64 years should undertake at least 150 minutes of moderate-intensity physical activity, or at least 75 minutes of vigorous-intensity physical activity, or some equivalent combination of moderate-intensity and vigorous-intensity aerobic physical activity, per week. In order to derive additional health benefits, adults should increase their moderate-intensity physical activity or equivalent to 300 minutes per week and should also engage in muscle-strengthening activities that involve all major muscle groups, which should be practised on two or more days a week. Similarly WHO physical activity guidelines recommends that adults aged 65 and above should undertake at least 150 minutes of moderate-intensity physical activity, or at least 75 minutes of vigorous-intensity physical activity, or some equivalent combination of moderate-intensity and vigorous-intensity aerobic physical activity, per week. As well, additional health benefits may be obtained by increasing their moderate-intensity physical activity, or equivalent to 300 minutes per week. People with reduced mobility should engage in physical activity that serves to emphasise and enhance functional balance and prevent falls, on three or more days a week. Muscle-strengthening activities that involve all major muscle groups should be done on two or more days a week.

In addition, the use of NMN, which is a molecule naturally present in the body, has many advantages. In particular, NMN does not pose any tolerance problem in patients. The use of NMN and of the composition according to the invention in fact does not induce any allergies. In addition, the use of NMN and of the composition according to the invention does not induce the adverse side effects frequently encountered with conventional treatments.

In particular, NMN also does not induce any phenomenon of physical or psychological dependence. NMN also does not induce any bone fragility or vulnerability to infections as is observed with the chronic administration of cortisone or its derivatives. The use of NMN and of the composition according to the invention for preventing and/or treating muscle, ligament or tendon pain or their combinations, and in particular muscle pain, resulting from physical activity, and in particular the practice of a sport, is therefore safe for patients.

The NMN and the composition according to the invention may be used for adults as also for children. NMN is indeed well tolerated by children. In the context of the invention, patients are deemed to be children if aged less than 18 years, and adults from the age of 18 onwards. Consequently, the invention is also of interest in treating muscle, ligament or tendon pain or their combinations in children.

The salts suitable for the implementation of the invention are obtained from an organic or inorganic base or acid of NMN. By way of example of salts, mention may be made of chlorides, bromides, fluorides, iodides, sulfates; as well as salts of sodium, potassium, magnesium, formate, acetate, propionate, butyrate, glutamate, aspartate, ascorbate, benzoate, carbonate, citrate, carbamate, gluconate, lactate, methyl bromide, methyl sulfate, nitrate, phosphate, diphosphate, succinate, sulfonate, trifluoromethanesulfonate, trichloromethanesulfonate, tribromomethanesulfonate, and trifluoroacetate. Preferably, the salt is a chloride.

In one particularly preferred embodiment, the NMN is in the form of a zwitterion. The term “zwitterion” is understood to refer to a molecular chemical species that possesses electrical charges of opposite signs and situated, in general, on non-adjacent atoms of the molecule.

The derivative of NMN may be selected from among dihydronicotinamide mononucleotide (denoted NMN-H), alpha-NMN;

a compound having the formula (I):

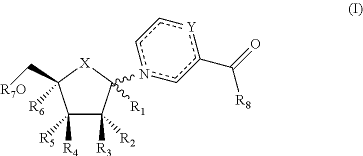

or one of the pharmaceutically acceptable: stereoisomers, salts, hydrates, solvates, or crystals thereof, in which:
X is selected from among O, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄ and R₅ are selected independently of one another, from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from a proteinogenic amino acid;
R₆ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;

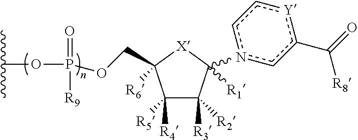

R₇ is selected from among H, P(O)R₉R₁₀, P(S)R₉R₁₀ and wherein n is an integer selected from 1 or 3; in which
R₉ and R₁₀ are selected independently of one another, from among OH, OR₁₁, NHR₁₃, NR₁₃R₁₄, a (C₁-C₈) alkyl, a (C₂-C₈) alkenyl, a (C₂-C₈)alkynyl, a (C₃-C₁₀) cycloalkyl, a (C₅-C₁₂) aryl, (C₁-C₈)alkyl aryl, (C₁-C₈) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —(CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)ₙC(O)O(C₁-C₁₅)alkyl, and —(CH₂)ₙC(O)O(C₁-C₁₅)alkyl aryl; wherein n is an integer selected from 1 to 8; P(O)(OH)OP(O)(OH)₂; halogen, nitro, cyano, C₁-C₆ alkoxy, C₁-C₆ haloalkoxy, —N(R₁₁ₐ)₂, C₁-C₆ acylamino, —COR_{11b}, —O COR_{11b}; NHSO₂(C₁-C₆ alkyl), —SO₂N(R₁₁ₐ)₂ SO₂; wherein each of R₁₁ₐ is independently selected from H and a (C₁-C₆) alkyl, and Ru_{11b} is independently selected from OH, C₁-C₆ alkoxy, NH₂, NH(C₁-C₆ alkyl) or N(C₁-C₆ alkyl)₂;
R₁₂ is selected from among H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the said aryl or heteroaryl groups are optionally substituted with one or two groups selected from among halogen, trifluoromethyl, C₁-C₆ alkyl, C₁-C₆ alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl, —(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among hydroxyl, (C₁-C₁₀) alkyl, (C₁-C₆) alkoxy, a halogen, a nitro, and a cyano; or
R₉ and R₁₀ form, together with the phosphorus atoms to which they are attached, a 6-membered ring in which —R₉—R₁₀— represents —CH₂—CH₂—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or

R₉ and R₁₀ form, together with the phosphorus atoms to which they are attached, a 6-membered ring in which —R₉—R₁₀— represents —O—CH₂—CH₂—CHR—O—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano;
R₈ is selected from among H, OR, NHR₁₃, NR₁₃R₁₄, NH—NHR₁₃, SH, CN, N₃, and halogen; wherein R₁₃ and R₁₄ are selected independently of one another, from among H, (C₁-C₈) alkyl, (C₁-C₈) alkyl aryl, and —CR_{B}R_{C}—C(O)—OR_{D} in which R_{B}and R_{C} are independently a hydrogen atom, a (C₁-C₆) alkyl, a (C₁-C₆) alkoxy, benzyl, indolyl, or imidazolyl; where the (C₁-C₆) alkyl and the (C₁-C₆) alkoxy may be optionally and independently of one another substituted by one or more of the halogen, amino, amido, guanidyl, hydroxyl, thiol, or carboxyl groups, and the benzyl group is optionally substituted by one or more halogen or hydroxyl groups; or R_{B} and R_{C} form, together with the carbon atom to which they are attached, a C₃-C₆ cycloalkyl group optionally substituted by one or more halogens, amino, amido, guanidyl, hydroxyl, thiol, and carboxyl; and R_{D} is a hydrogen, a (C₁-C₆) alkyl, a (C₂-C₆) alkenyl, a (C₂-C₆) alkynyl, or a (C₃-C₆) cycloalkyl;
Y is selected from among CH, CH₂, C(CH₃)₂ and CCH₃;
[symbol] represents a single or a double bond along Y; and
[symbol] represents the alpha or beta anomer depending on the position of R₁;
   or

a compound having the formula (Ia):

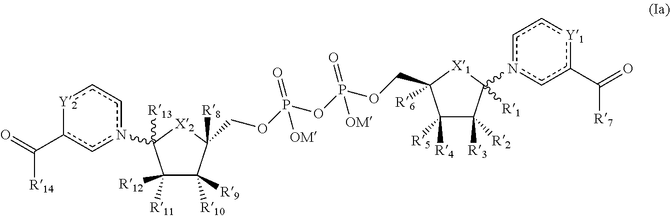

or one of the: stereoisomers, salts, hydrates, solvates, or crystals thereof, in which:

X′₁ and X^{′}2 are independently selected from among O, CH₂, S, Se, CHF, CF₂, and C═CH₂;

R′₁ and R′13 are independently selected from among H, azido, cyano, a C1-C8 alkyl, a C1-C8 thio-alkyl, a C1-C8 heteroalkyl, and OR, wherein R is selected from H and a C1-C8 alkyl;

R′₂, R′₃, R′₄, R′₅, R′₉, R′₁₁, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH2 group; wherein R_{AA} is a side chain selected from a proteinogenic amino acid;

R′₆ and R′₈ are independently selected from among H, an azido, a cyano, a C₁-C₈ alkyl and OR, wherein R is selected from H and a C₁-C₈ alkyl;

R′₇ and R′₁₄ are independently selected from among H, OR, NHR, NRR′, NH—NHR, SH, CN, N₃ and a halogen; wherein R and R′ are independently selected from H and a (C₁-C₈) alkyl aryl;

Y′₁ and Y′₂ are independently selected from among CH, CH₂, C(CH₃)₂, or CCH₃;

M′ is selected from H or a suitable counter ion;

[symbol] represents a single or double bond depending on Y′₁ and Y′₂; and; [symbol] represents an alpha or beta anomer depending on the position of R′₁ and R′₁₃;

and combinations thereof.

Within the meaning of the invention, M′ may be an internal or external counter ion.

In a first preferred embodiment, the pharmaceutically acceptable derivative is the compound having the formula (I).

In one variant of the first embodiment, X represents an oxygen.

In one variant of the first embodiment, R₁ and R₆ each independently of one another represent a hydrogen.

In one variant of the first embodiment, R₂, R₃, R₄ and R₅ each independently of one another represent a hydrogen or an OH.

In one variant of the first embodiment, Y represents a CH.

In one variant of the first embodiment, Y represents a CH₂.

In one variant of the first embodiment, R₇ represents a hydrogen.

In one variant of the first embodiment, R₇ represents P(O)(OH)₂.

In one variant of the first embodiment,

X represents an oxygen; and/or

R₁ and R₆ each independently represent a hydrogen; and/or

R₂, R₃, R₄ and R₅ each independently represent a hydrogen or R₂, R₃, R₄ and R₅ independently represent OH; and/or

Y represents a CH or a CH₂; and/or

R⁷ represents P(O)R₉R₁₀, in which R₉ and R₁₀ are independently selected from among OH, OR₁₁, NHR₁₃, NR₁₃R₁₄, C₁-C₈ alkyl, C₂-C₈ alkenyl, C₂-C₈ alkynyl, C₃-C₁₀ cycloalkyl, C₅-C₁₂ aryl, C₁-C₈ aryl alkyl, C₁-C₈ alkyl aryl, C₁-C₈ heteroalkyl, C₁-C₈ heterocycloalkyl, heteroaryl, and NHCR_{A}R_{A′}C(O)R₁₂.

In one particularly preferred variant of the first embodiment, the compound of the invention is selected from among the compounds having the formula IB to IJ:

TABLE 1

| Compounds | |
|---|---|
| (Anomers) | Structure |
| I-B (alpha) | 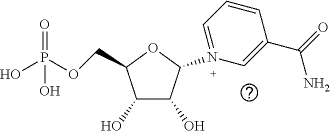 |
| I-C (beta) | 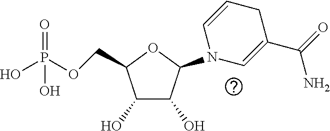 [SMILES: NC(=O)C1=CN([C@@H]2O[C@H](COP(=O)(O)O)[C@@H](O)[C@H]2O)C=CC1] |
| I-D (alpha) | 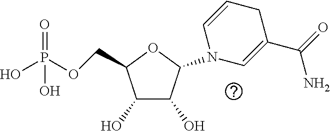 [SMILES: NC(=O)C1=CN([C@H]2O[C@H](COP(=O)(O)O)[C@@H](O)[C@H]2O)C=CC1] |
| I-E (beta) | 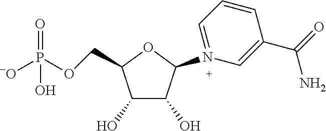 |
| I-F (alpha) | 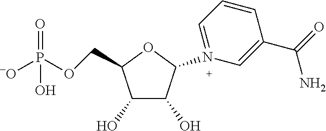 |
| I-G (beta) | 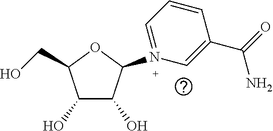 |
| I-H (alpha) | 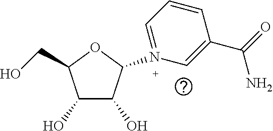 |
| I-I (beta) | 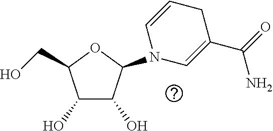 [SMILES: NC(=O)C1=CN([C@@H]2O[C@H](CO)[C@@H](O)[C@H]2O)C=CC1] |
| I-J (alpha) | 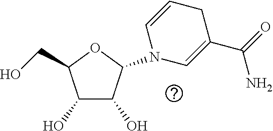 [SMILES: NC(=O)C1=CN([C@H]2O[C@H](CO)[C@@H](O)[C@H]2O)C=CC1] |

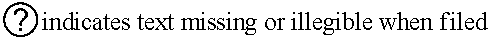

The pharmaceutically acceptable derivative of NMN may be alpha-NMN (Compounds I-B or I-F) or dihydronicotinamide mononucleotide (NMN-H) (Compounds I-D or I-C), and combinations thereof.

In a second preferred embodiment, the pharmaceutically acceptable derivative is the compound having the formula (Ia).

In one variant of the second embodiment, X′1 and X′2 each independently represent an oxygen.

In one variant of the second embodiment, R′7 and R′14 each independently represent an NH₂.

In one variant of the second embodiment, R′1 and/or R′13 each independently represent a hydrogen.

In one variant of the second embodiment, R′6 and/or R′8 each independently represent a hydrogen.

In one variant of the second embodiment, R′2, R′3, R′4, R′5, R′9, R′10, R′11, and R′12 each independently represent a hydrogen.

In one variant of the second embodiment, R′2, R′3, R′4, R′5, R′9, R′10, R′11, and R′12 each independently represent an OH.

In one variant of the second embodiment, Y′1 and Y′2 each independently represent a CH.

In one variant of the second embodiment, Y′1 and Y′2 each independently represent a CH2.

In one variant of the second embodiment, the compound according to the invention is selected from among the compounds having the formula Ia-A to Ia-I:

TABLE 2

| Compounds | |
|---|---|
| (Anomers) | Structure |
| Ia-A (beta, beta) | 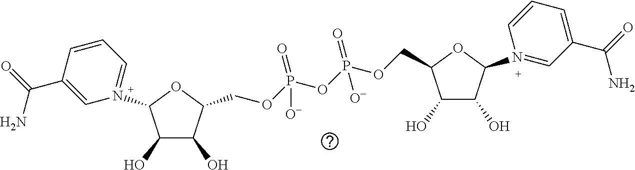 |
| Ia-B (beta, alpha) | 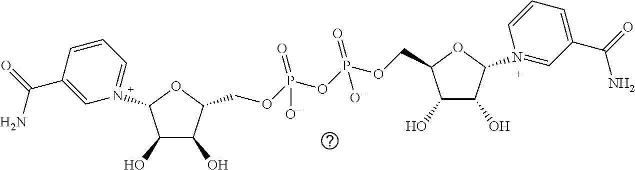 |
| Ia-C (alpha, alpha) | 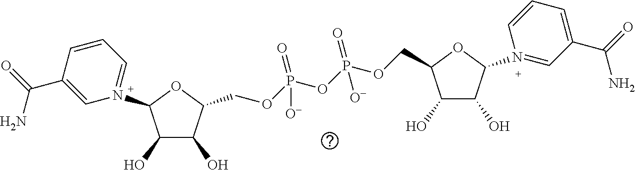 |
| Ia-D (beta, beta) | 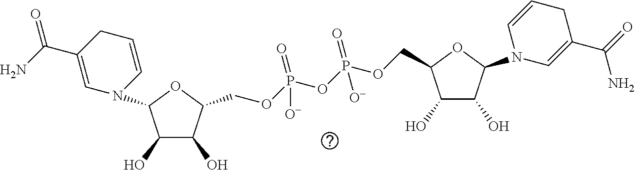 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| Ia-E (beta, alpha) | 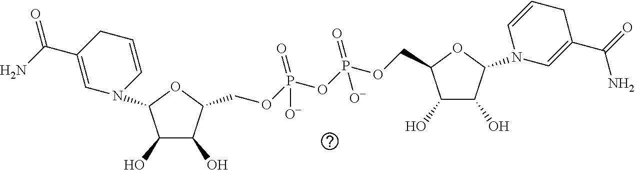 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-F (alpha, alpha) | 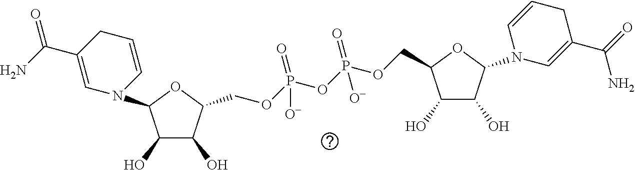 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-G (beta, beta) | 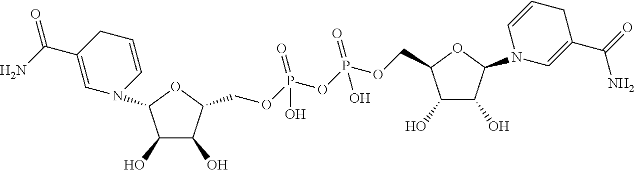 [SMILES: NC(=O)C1=CN([C@@H]2O[C@H](COP(=O)(O)OP(=O)(O)OC[C@H]3O[C@@H](N4C=CCC(C(N)=O)=C4)[C@H](O)[C@@H]3O)[C@@H](O)[C@H]2O)C=CC1] |
| Ia-H (beta, alpha) | 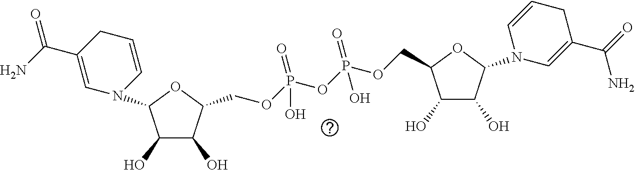 [SMILES: NC(=O)C1=CN([C@@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |
| Ia-I (alpha, alpha) | 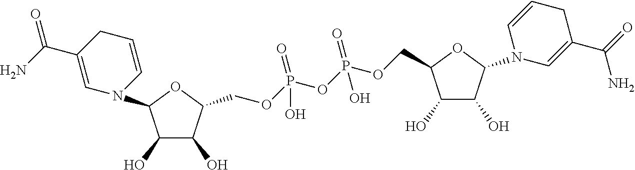 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

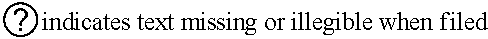

Preferably, the compound having the formula Ia is selected from among the compounds Ia-B, Ia-C, Ia-E, Ia-F, Ia-H and Ia-I, and combinations thereof.

By reducing the use of therapies that are used in a conventional manner, or indeed even replacing them, the present invention therefore makes it possible to avoid, or at the very least to reduce, the use of conventional treatments for muscle, ligament, or tendon pain or combinations thereof, and therefore to avoid, or at the very least to reduce, the occurrence of adverse side effects linked to these therapies.

Use

According to the present invention, NMN, the pharmaceutically acceptable derivatives or the pharmaceutically acceptable salts thereof, as well as the compositions that comprise the same, are used to prevent and/or treat muscle, ligament or tendon pain or combinations thereof resulting from a/some physical activity.

The physical activity that could induce muscle, ligament, or tendon pains or combinations thereof depends partly on the physical condition of the person and the nature of the physical activity.

Physical activity may be undertaken for recreational purposes, for professional purposes or for sports. By way of non-limiting examples of a physical activity for recreational purposes, mention may be made of walking, shopping, DIY activities, assembling furniture, gardening, fishing, and cooking. Physical activity that can cause muscle, ligament, or tendon pain and combinations thereof may, for example, be assembly line work, work requiring physical manipulations as performed in occupations such as physiotherapist, osteopath, nurse, caregiver, stretcher bearer/hospital orderly, firefighter, paramedic-first responder, janitorial personnel, storekeeper, salesman, security guard and others.

The intensity of the various different forms of physical activity varies from person to person. In order for any activity to be beneficial from the cardiorespiratory endurance perspective, it must be performed in increments of at least 10 minutes. In its Global Physical Activity Questionnaire, the WHO provides examples of intense or moderate physical activity, whether for work or leisure. For example, vigorous-intensity physical activity requires a substantial increase in breathing or heart rate, such as carrying or lifting heavy loads, working on a construction site, doing masonry work, running, or playing football for at least 10 minutes continuously at a stretch. Moderate-intensity physical activity may be brisk walking or carrying light loads, swimming, biking, or playing volleyball for at least 10 minutes continuously at a stretch. In terms of physical activity, the WHO also takes into account the mode of transport from one place to another, such as walking or cycling. Light-intensity physical activity may be, for example, slow walking or washing dishes; moderate-intensity physical activity may be brisk walking or aquatic exercise/water aerobics, and vigorous-intensity physical activity may be jogging or tennis.

There are 4 discernable levels of activity intensity, the physical intensity effectively being dependent on the perceptions of each individual:

Low intensity activities—such as driving a car, tidying up, food preparation or cooking—do not induce shortness of breath or sweating. The perceived exertion level is rated at 3 or 4 on a scale of 0 to 10.

Moderate intensity activities—such as brisk walking, running (less than 8 km/h), cycling (approximately 15 km/h), or climbing stairs—induce moderate shortness of breath and slight sweating. The perceived exertion level is rated at 5 or 6 on a scale of 0 to 10. Holding a conversation is possible.

High-intensity activities—such as walking briskly or uphill, running, cycling (about 20 km/h) or lifting/moving heavy loads—induce marked shortness of breath and profuse sweating. The perceived exertion level is rated at 7 or 8 on a scale of 0 to 10. Holding a conversation is difficult.

Very high intensity activities—such as running (9 to 18 km/h), cycling (over 25 km/h), jumping rope—induce severe shortness of breath and very abundant sweating. The perceived exertion level is rated at more than 8 on a scale of 0 to 10. Holding a conversation is impossible.

Preferably, the muscle, ligament, or tendon pain or combinations thereof results from the practise of a sport.

By way of examples of a sport causing muscle, ligament or tendon pains and combinations thereof, on a non-exhaustive or non-limiting basis the following may be mentioned: hiking, Nordic walking, fitness walking, running, horse riding, athletics, dance, gymnastics, racket sports, combat sports, team sports, water sports, extreme sports. By way of examples of racket sports, mention may be made, in a non-exhaustive manner, of tennis, badminton, squash, table tennis and tennis. By way of examples of combat sports, mention may be made of martial arts, French boxing, English boxing, Thai boxing, fencing, wrestling, capoeira, catch (professional wrestling), savate. By way of examples of team sports, mention may be made, in a non-exhaustive manner, of football, handball, volleyball, basketball, rugby, polo and water polo. By way of examples of water sports, mention may be made of kayaking, sailing, windsurfing, diving, canoeing. By way of examples of extreme sports, mention may be made, in a non-exhaustive manner, of skydiving, paragliding, kitesurfing, surfing, canyoning, mountaineering, and rock climbing.

NMN, a derivative or a salt thereof, as well as the compositions that comprise the same according to the invention may in fact be used to relieve muscle, ligament or tendon pains or combinations thereof, linked to the practise of sports, and in particular muscle pain, without resorting to the use of conventional therapies.

In particular, NMN, a pharmaceutically acceptable derivative thereof, or a pharmaceutically acceptable salt thereof, as well as compositions that comprise the same may be used in the treatment and/or prevention of muscle, ligament or tendon pain or combinations thereof, in particular muscle pain, induced by physical activity, in mammals and preferably in humans.

In the context of the present invention, the muscle, ligament, or tendon pains or combinations thereof, result solely from physical activity, preferably from the practise of a sport. Such pains are localised or diffuse and their treatment does not call for surgical interventions. In particular, such pains may result from: excessive practise of a physical activity, and in particular of a sport; a badly executed action/manoeuvre or improper movement; bad posture while practising a physical activity and in particular sports activity; lack of stretching after a physical activity and in particular sports activity; the use of inappropriate equipment while practising a physical activity and in particular sports activity; and/or lack of rest.

The muscle, ligament, or tendon pains or combinations thereof concern all the muscles in the human body. Preferably, the muscle, ligament, or tendon pains or combinations thereof concern the thigh, calf, foot, hip, gluteal muscles, trapezius muscle, shoulder, abdominals, biceps, triceps, forearm muscles, neck muscles, foot muscles, Achilles tendon, hand, or combinations thereof.

Preferably, the pain is a muscle pain. Within the meaning of the present invention, the muscle pain affects the skeletal striated muscles, and not the smooth muscles or cardiac muscle. The muscle pain may be selected from among: soreness, contracture, cramping, elongation (pulled/strained muscles), muscle contusion, muscle tearing, partial or complete rupture of muscle fibres, or combinations thereof.

The term “soreness” refers to the prolonged and involuntary contraction of one or more muscles due to exertion of a physical effort. The soreness is the consequence of the muscle performing anaerobic work, that is to say without supply of oxygen, with production of lactic acid and accumulation of cellular waste due to the metabolism in lack of oxygen. The soreness appears after the exertion of effort and can persist for 1 to 7 days. Muscle contracture and cramp correspond to involuntary muscle contractions of a few muscle fibres of a muscle or a muscle group, and appear during exercise. Contracture differs from cramp in that it persists over a far longer period. In fact, the cramp persists for only a few minutes while the contracture can persist for 5 to 6 days, if the muscle is left to rest. Cramp and contracture are usually not associated with muscle injury (lesion).

The term “elongation” (pulled/strained muscles) is understood to refer to the traumatic stretching of a muscle or muscle group. The term “muscle contusion” is understood to refer to an injury to the muscle or a muscle group, without breaking the skin and characterised by discoloration of the skin and swelling.

Advantageously, the muscle pain may be classified under one of the categories M62.1, M62.4, M62.6, R25.2 of the International Classification of Diseases ICD-10 (10^{th} revision, 2019 edition) and combinations thereof.

Advantageously, the ligament pain may be a sprain, a partial or complete tear of the ligament or combinations thereof.

A sprain is a stretching of the ligaments. When it is benign, the sprain may also be referred to by the term “strain”, although this term has been abandoned by the medical profession.

Advantageously, the tendon pain is tendinitis, tenosynovitis, bursitis, or combinations thereof.

The term “tendonitis” is understood to refer to the inflammation of the tendon. The term “tenosynovitis” refers to inflammation of the tendon and surrounding synovial sheath. The term “bursitis” is understood to refer to an inflammation of the periarticular serous bursa and tendons, often caused by repetitive stress on the joint or the surrounding tendons.

Advantageously, the tendon pain may in particular be classified under category M65, preferably M65.2, M65.3, M65.4, M65.5, M65.6, M65.7, M65.8, M65.9, M66, M67, M70.6, M75.1 (Rotator cuff syndrome), M75.2, M75.3, M76, M77 of the ICD-10 classification (10^{th} revision, 2019 edition) and combinations thereof.

According to the ICD10 (2019 revision), the pain may occur during an event whereof the activity code is classified as code 0, that is to say “While practising a sport”, or as code 1, that is to say “While participating in a game and leisure activities’.

The Nicotinamide mononucleotide (NMN), a pharmaceutically acceptable derivative thereof, or a pharmaceutically acceptable salt thereof, as well as compositions that comprise the same also make it possible to reduce muscle, ligament or tendon stiffness or combinations thereof, and/or to improve muscle, ligament, tendon function or combinations thereof. The term “muscle stiffness” is understood to refer to the difficulty of the muscle to relax. “Muscle function” refers to the muscle's ability to contract. The term “ligament or tendon stiffness” is understood to refer to the difficulty of the ligament or tendon to stretch so as to enable movement. The term “function of the ligament or tendon” is understood to refer to the ability of the ligament or tendon to stretch normally, that is to say in a non-exaggerated or limited manner, in order to enable movement.

However, in the context of the present invention, the muscle, ligament, or tendon pain or combinations thereof is not due to one of the pathologies selected from the following: a tumour, arthritis (classes M00 to M09 and M11 to M14 of the ICD -10), gout (ICD-10 class M10), osteoarthritis (ICD-10 classes M15 to M19), a joint deformity (ICD-10 classes M20 and M21), a connective tissue disease (ICD-10 classes M30 to M36), dorsopathy (ICD-10 classes M40 to M54), a neurodegenerative disease, neuropathy, a genetic disease, an autoimmune disease, myopathy (ICD-10 classes M60-M63), osteopathy (ICD-10 classes M80 to M90), osteoporosis, chondropathy (ICD-10 classes M91 to M94), vasculopathy, a viral infection, a fungal infection, a bacterial infection, a parasite, adverse side effects of a medication, a surgical procedure, a medical examination, calcification, trauma (unless induced by a physical activity), a malformation or combinations thereof.

The term “neurodegenerative disease” is understood to refer to a progressive pathology which affects the brain or more generally the nervous system, resulting in the death of nerve cells. The term “neuropathy” is understood to refer to all the conditions/disorders essentially of the peripheral nervous system, that is to say of the motor and sensory nerves and of the limbs, of the nerves of the autonomic nervous system which control the organs as well as more rarely of the central nervous system. The neuropathies may be caused by—but are not limited to—abuse of alcohol, medications, diabetes, a viral infection, an injury that has damaged a nerve, or may result from an unknown cause. The term “genetic disease” is understood to refer to a disease due to one or more anomalies on one or more chromosomes which lead to a malfunction of certain cells in the body. A genetic disease may be due to a deletion or a mutation in a gene leading to the formation of an inactive or malformed protein.

An autoimmune disease is a disease in which the immune system is overactivated and attacks normal human cells.

A myopathy is a degenerative muscular disease characterised by a decrease in the strength of the affected muscles and a variable degree of atrophy. These are most often hereditary diseases. The term “chondropathy” is understood to refer to a disease affecting the cartilage and may be due to pressure that is excessive or poorly distributed over the cartilage. It could manifest as a softening of the cartilage. The term “osteoporosis” is understood to refer to a rarefaction of bone tissue and may result from menopause or aging, or be idiopathic. The term “Vasculopathy” is understood to refer to a pathology affecting the arterial or venous vessels.

The term “trauma” is understood to refer to a fracture, dislocation, subluxation, or combinations thereof.

There exist various different scales for measuring pain. Such measurement scales are for example listed in the document provided by the Haute Autorité de Santé/French National High Authority for Health (https://www.has-sante.fr/upload/docs/application/pdf/2019-02/liste echelles douleur 2019.pdf). Among these the following noteworthy scales may be mentioned: the Visual Analogue Scale (VAS) of pain, the numerical scale, the simple verbal scale. The visual analogue scale may be presented in its paper form or in its “mechanical” form, that is to say as a type of ruler. The conventional form features a 100 mm long horizontal line. The patient indicates the level of their pain by drawing a line over the printed line (paper form) or by moving a cursor along this line (mechanical ruler). It has a white background and no words other than those at the ends. Some scales have been developed for particular categories of the population. For example, the Doloplus and Algoplus scales have been developed specifically for the elderly.

Mode of Administration and Galenic Form

NMN, a pharmaceutically acceptable derivative thereof, or a pharmaceutically acceptable salt thereof, and compositions that comprise the same are preferably intended to be administered via the topical route. The term “topical route” is understood to refer to the form of administration of a composition or a substance at a site or on an external surface of the body, such as the skin or the mucous membranes.

The galenic or pharmaceutical dosage forms that are suitable for implementing the invention are a gel, a solution, a water-in-oil emulsion, an oil-in-water emulsion, an oil, a cream, an ointment/salve, or a liniment.

The term “solution” is understood to refer to a liquid galenic form used for the administration of at least one active ingredient that is obtained by dissolving the various ingredients in a liquid phase so as to form only one homogeneous phase.

The term “emulsion” is understood to refer to a heterogeneous mixture of two immiscible liquid substances, one thereof being dispersed in the form of small droplets in the other. An emulsion is used to mix two liquids which do not mix spontaneously (immiscible), like water and oil. An emulsion may be obtained by using specific operations (agitation, mixing, addition of some active ingredients). An emulsion has an appearance that is macroscopically homogeneous, but microscopically heterogeneous. One of the substances will therefore be dispersed in the second substance in the form of droplets. The mixture is able to remain stable thanks to a third ingredient referred to as emulsifier (speed or kinetics of evolution of the mixture is almost zero). A “water-in-oil emulsion”, denoted “water/oil”, is composed of an aqueous phase dispersed in an oily phase. An “oil-in-water emulsion”, denoted “oil/water”, is composed of an oily phase dispersed in an aqueous phase.

The term “cream” is understood to refer to a semi-solid preparation intended to be administered for topical use.

The term “ointment/salve” is understood to refer to a semi-solid preparation intended to be applied over the skin.

The term “liniment” is understood to refer to a liquid pharmaceutical form, conventionally comprising fatty substances such as oils, intended to be used by rubbing (friction) action.

The term “gel” is understood to refer to a solid material, possibly ductile, consisting of a three-dimensional network of macromolecules surrounded by liquid. A composition in the form of a gel penetrates well and rapidly into the skin and also serves to provide an anesthetic sensation of freshness.

In one preferred embodiment, the gel may be a hydrophobic gel or a hydrophilic gel. Advantageously, the gel is a hydrophilic gel.

In one particularly preferred embodiment, the composition according to the invention is in the form of a water-in-oil emulsion or an oil-in-water emulsion, more preferably in the form of an oil-in-water emulsion (denoted oil/water or O/W).

NMN is very hydrophilic and therefore dissolves better in aqueous phases.

Advantageously, the NMN, a pharmaceutically acceptable derivative thereof, or a pharmaceutically acceptable salt thereof, as well as the composition comprising the same, is intended to be administered between 1 and 10 times per day, preferably between 1 and 5 times per day, in a more preferred manner between 1 and 3 times per day.

In one particularly preferred embodiment, the NMN, a pharmaceutically acceptable derivative thereof, or a pharmaceutically acceptable salt thereof, or the composition comprising the same is may be administered twice a day.

Therapeutic Combinations

NMN, a pharmaceutically acceptable derivative thereof, a pharmaceutically acceptable salt thereof, as well as compositions that comprise the same may also be used in combination with at least one other therapeutic agent, in particular the therapeutic agents conventionally used in the prevention and/or treatment of muscle, ligament or tendon pain or their combinations, induced by physical activity.

Among the therapeutic agents that may be combined with the invention, mention may be made of an analgesic, a non-steroidal anti-inflammatory drug, cortisone, a cortisone derivative, a muscle relaxant, arnica, salicylic acid, aescin, capsaicin, zucapsaicin, tolazoline, dimethyl sulfoxide, idrocilamide, or combinations thereof.

Advantageously, the analgesic may be selected from among paracetamol, nefopam, ketanin, tetrahydrocannabinol, cannabinoids, aspirin, methyl salicylate, diflunisal, salicylamide, codeine, alfentanil, carfentanil, dihydrocodeine, codeinone, tramadol, morphine, morphinone, buprenorphine, fentanyl, acetyl fentanyl, remifentanil, sufentanil, heroin, hydromorphone, nalbuphine, oxycodone, hydroxycodone, oxymorphone, laudanum, methadone, pethidine, dextropropoxyphene, endorphin, tapentadol, thebaine, vicodin, and combinations thereof.

Advantageously, the non-steroidal anti-inflammatory drug may be selected from among ibuprofen, ketoprofen, naproxen, ketorolac, alminoprofen, aceclofenac, mefenamic acid, niflumic acid, tiaprofenic acid, celecoxib, rofecoxib, valdecoxib, parecoxib, dexketoprofen, diclofenac, etodolac, etoricoxib, fenoprofen, flurbiprofen, indomethacin, meloxicam, nabumetone, piroxicam, sulindac, tenoxicam, nimesulide, and combinations thereof.

The cortisone derivative may be selected from among betamethasone, ciprofloxacin, cortivazol, dexamethasone, fludrocortisone, methylprednisolone, prednisolone and triamcinolone, and combinations thereof.

Arnica is the plant known as Arnica montana. It may be incorporated into the composition according to the invention or be used in combination with NMN, a pharmaceutically acceptable derivative or salt thereof, or the composition according to the invention. Arnica may in particular be used as an infusion or in the form of an extract incorporated into a salve, an ointment, a gel or any appropriate galenic form suitable for topical application.

Aescin, also referred to as horse chestnut or beta-aescin, may be applied topically to reduce pain in combination with the use of NMN or compositions that comprise the same as described.

Capsaicin and zucapsaicin are alkaloids found in the pepper family. They are preferably administered locally.

Advantageously, the muscle relaxant may be selected from among locally acting muscle relaxants, centrally acting muscle relaxants and carbamic acid esters and derivatives thereof.

Muscle relaxants are a class of medicinal products used to relax skeletal striated muscles. Muscle relaxants act either locally on the muscle or by central action. Centrally acting muscle relaxants act on the central nervous system in the spinal cord or brain.

By way of non-limiting examples of muscle relaxants that may be used in combination with the invention, mention may in particular be made of baclofen, quinine, mephenesin, tizanidine, tetrazepam, thiocolchicoside, acetyl hexapeptide-8, μ-conotoxin CnIIIc (mu-conotoxin CnIIIc), dipeptide diaminobutyroyl benzylamide diacetate as well as locally used botulinum toxin, and combinations thereof.

Acetyl hexapeptide-8 is also referred to as argireline and is registered under CAS number: 616204-22-9. Its action mimics the action of botulinum toxin. The μ-conotoxin CnIIIc (or mu-conotoxin CnIIIc) is a derivative of botulinum toxin: it enables the blocking of the Nav sodium channels and nicotinic acetylcholine receptors denoted as nAChr. The μ-conotoxin CnIIIc (or mu-conotoxin CnIIIc) is registered under CAS number: 936616-33-0 and under UNIPROT number |1SB07. Dipeptide diaminobutyroyl benzylamide diacetate is registered under CAS number: 823202-99-9. It is used to reduce muscle contraction. Argireline, μ-conotoxin CnIIIc, and dipeptide diaminobutyroyl benzylamide diacetate are preferably intended to be administered via the topical route.

In one particularly preferred embodiment, the muscle relaxant acts locally on the muscle. Locally acting muscle relaxants may be administered via either the intramuscular or topical routes.

Advantageously, the carbamic acid ester may be methocarbamol.

The at least one other additional therapeutic agent may be administered via either topical or oral routes, or by injection. More precisely, the at least one other therapeutic agent may be administered by the route by which it is conventionally administered.

The at least one other therapeutic agent may also be administered concomitantly with, or at different times from the NMN, a pharmaceutically acceptable salt thereof, or a pharmaceutically acceptable derivative thereof, or from the composition according to the invention.

Although the NMN and the compositions that comprise the same make it possible to replace the use of conventional therapies, in one embodiment it may be envisaged to combine the invention with conventional therapies for the prevention and/or treatment of muscle, ligament and/or tendon pain. Indeed, such a combination makes it possible to reduce the dose and/or the frequency of administration of the conventional therapies and consequently to avoid or at the very least reduce the side effects associated with these conventional therapies.

The composition and the compounds according to the invention may be administered simultaneously, separately or sequentially with the at least one additional therapeutic agent. The term “simultaneously” is understood to indicate that two agents are administered at the same time. The term “separately”, is understood to indicate that the time interval between the administration of the first agent and that of the second is significant and at least one hour. The term “sequentially” is understood to indicate that the two agents are administered one after the other within a timeframe such that they are both available to act therapeutically over the same time period. The optimum time interval between administration of the two agents will vary depending on the precise nature of the method of administration of the compounds or compositions of the invention.

Compositions

The compositions according to the invention may comprise nicotinamide mononucleotide, a pharmaceutically acceptable derivative thereof, or a pharmaceutically acceptable salt thereof, and at least one pharmaceutically acceptable excipient for the topical administration thereof for use in the prevention and/or the treatment of a muscle, ligament or tendon pain or their combination, induced by physical activity.

Preferably, the compositions according to the invention are intended to be administered topically.

Such compositions are particularly useful for relieving muscle, ligament or tendon pain or combinations thereof, induced by physical activity, in particular resulting from the practice of a sport as described in the description.

In the context of the present invention, an “excipient” refers to any substance other than the NMN that is in the composition and has no therapeutic effect. The excipient does not interact chemically with the NMN or any other additional therapeutic agent.

The excipient may be selected from among a bulking agent, a lubricant, a flavouring agent, a colouring agent, an emulsifier, a compression agent, a diluent, a preservative, a gelling agent, a plasticiser, a surfactant, or combinations thereof. A person skilled in the art would know how to determine the excipient to be selected based on the galenic form that they would have selected.

The composition according to the invention may be a pharmaceutical composition.

Advantageously, the composition according to the invention may comprise NMN, one of its salts or one of its pharmaceutically acceptable derivatives, in an amount of between 0.05% and 15% by weight, preferably between 1 and 10% by weight, more preferably between 3 and 5% by weight based on the total weight of the composition. Most preferably, the composition according to the invention comprises 5% of NMN, of a pharmaceutically acceptable derivative or salt thereof.

In one variant of interest, the composition according to the invention may also comprise at least one other additional therapeutic agent as defined above for use thereof in the prevention and/or treatment of a muscle, ligament, or tendon pain or combinations thereof, induced by physical activity as discussed above.

Method for Preparing the Compounds Having the Formula (I) and (IA)

The compounds having the formula (I) or the formula (Ia) may be prepared according to any method well known to the person skilled in the art.

Compound Preparation Method for Preparing the Compounds Having the Formula (I)

The compounds having the formula (I) may in particular be prepared according to the methods described in the international patent application WO 2017/024255A1, and the patent U.S. Pat. No. 10,611,790 B2, as well as according to the method described below.

In particular, the compounds having the formula (I) disclosed herein may be prepared as described here below from the substrates A-E. It is to be understood by the person skilled in the art that these reaction schemes are by no means intended to be limiting and that variations thereto may be made without departing in spirit and scope from the present invention.

According to one embodiment, the invention relates to a compound preparation method for preparing the compounds having the formula (I) as described here above.

The method involves, in a first step, the mono-phosphorylation of a compound having the formula (A), in the presence of phosphoryl chloride and a trialkyl phosphate, so as to thereby yield the phosphorodichloridate having the formula (B),

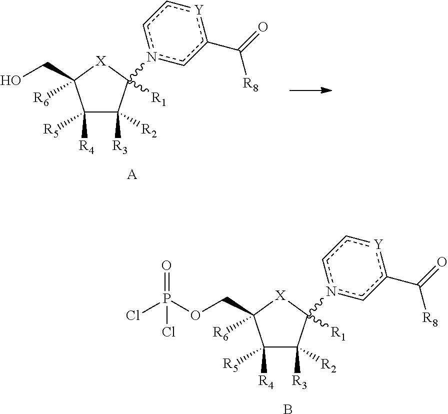

in which X, R₁, R₂, R₃, R₄, R₅, R₆, R₈, Y, [symbol] and [symbol] are as defined here above for the compounds having the formula (I).

In a second step, the phosphorodichloridate having the formula (B) is hydrolysed so as to thereby yield the phosphate having the formula (C),

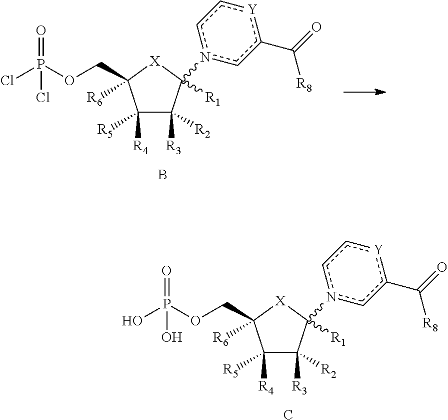

in which X, R₁, R₂, R₃, R₄, R₅, R₆, R₈, Y, [symbol] and [symbol] are as defined here above for the compounds having the formula (I).

According to one embodiment, the compound having the formula (A) is synthesised by means of various methods known to the person skilled in the art.

According to one embodiment, the compound having the formula (A) is synthesised by reaction of the pentose having the formula (D) with a nitrogenous derivative having the formula (E), in which R, R₂, R₃, R₄, R₅, R₆, R₇, Y, are as described here above for the compounds having the formula I, so as to thereby yield the compound having the formula (A-1) which is then selectively deprotected in order to give the compound having the formula (A),

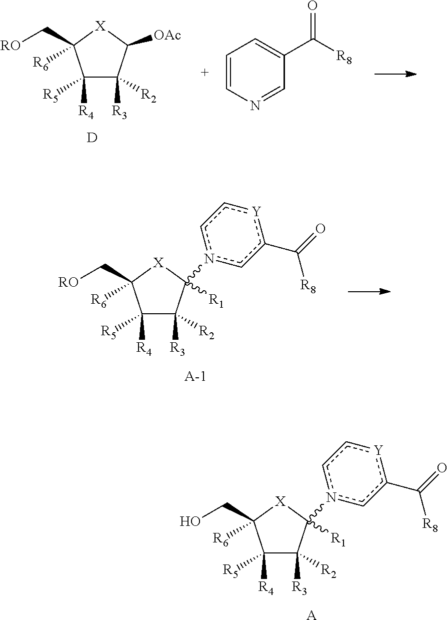

in which X, R₁, R₂, R₃, R₄, R₅, R₆, R₈, Y, [symbol] and [symbol] are as defined here above for the compounds having the formula (I).

According to one embodiment, R is a suitable protecting group known to the person skilled in the art.

In one embodiment, the protecting group is selected from among triarylmethyls and/or silyls. Without limitation, some examples of triarylmethyl include trityl, monomethoxytrityl, 4,4′-dimethoxytrityl, and 4,4′,4″-trimethoxytrityl groups. Without limitation, some examples of silyl groups include trimethylsilyl, tert-butyldimethylsilyl, triisopropylsilyl, tert-butyldiphenylsilyl, tri-iso-propylsilyloxymethyl, and [2-(trimethylsilyl)ethoxy]methyl.

According to one embodiment, any hydroxyl group attached to the pentose is protected by an appropriate protecting group known to the person skilled in the art.

The selection and exchanging of the protecting groups is well within the scope of knowledge and expertise of the person skilled in the art. The protecting groups may also be removed by methods well known to the person skilled in the art, for example, with an acid (for example, an inorganic or organic acid), a base or a fluoride source.

In one preferred embodiment, the nitrogenous derivative having the formula (E) is coupled to the pentose having the formula (D) by a reaction in the presence of a Lewis acid so as to thereby yield the compound having the formula (A-1). Without limitation, some examples of Lewis acids include Trimethylsilyl Trifluoromethanesulfonate (TMSOTf), BF₃.OEt₂, TiCl₄ and FeCl₃.

In one embodiment, the method of the present invention additionally also comprises a reduction step of reducing the compound having the formula (A) by various methods well known to the person skilled in the art, so as to thereby yield the compound having the formula (A′) in which is CH₂, and R₁, R₂, R₃, R₄, R₅, R₆, R₈, Y, [symbol] and [symbol] are as defined here above for the compounds having the formula (I).

In one particular embodiment, the present invention relates to a compound preparation method for preparing the compounds having the formula I-A, I-C, I-E, I-G.

In a first step, the nicotinamide having the formula E is coupled to the ribose tetraacetate having the formula D by a coupling reaction in the presence of a Lewis acid, so as to thereby yield the compound having the formula A-1::

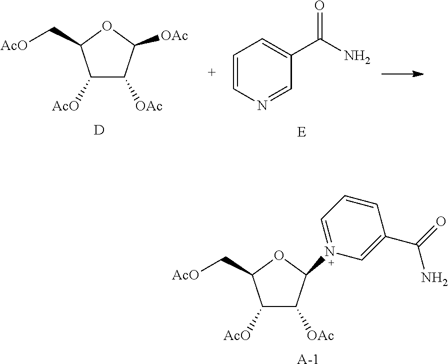

In a second step, an ammoniacal treatment of the compound having the formula A-1 is carried out, so as to thereby yield the compound having the formula I-A:

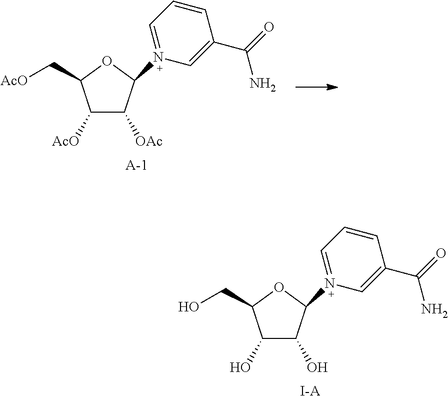

In a third step, the mono-phosphorylation of the compound having the formula I-A, in the presence of phosphoryl chloride and a trialkyl phosphate, thereby yields the phosphorodichloridate having the formula I-A′:

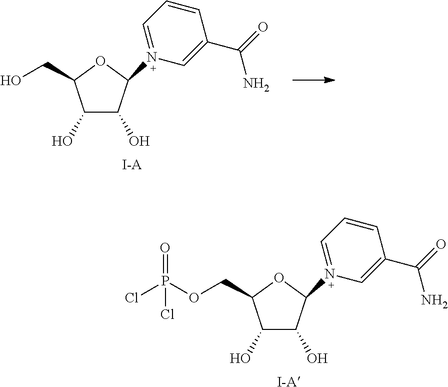

In a fourth step, the phosphorodichloridate having the formula B is hydrolysed so as to thereby yield the compound having the formula I-C:

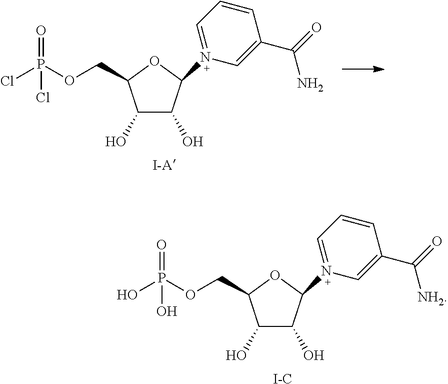

In one embodiment, a reduction step of reducing the compound having the formula I-A is carried out, so as to thereby yield the compound having the formula I-E.

The compound having the formula I-E is then mono-phosphorylated as described in the fourth step and hydrolysed so as to thereby yield the compound having the formula I-G.

According to one embodiment, the compounds having the formula (I) are selected from compounds I-A to I-H in the table below:

TABLE 1

| Compounds | |
|---|---|
| (Anomers) | Structure |
| I-A (beta) | 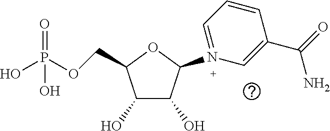 |
| I-B (alpha) | 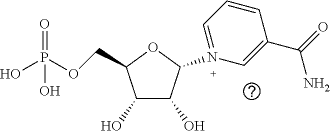 |
| I-C (beta) | 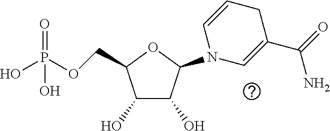 [SMILES: NC(=O)C1=CN([C@@H]2O[C@H](COP(=O)(O)O)[C@@H](O)[C@H]2O)C=CC1] |
| I-D (alpha) | 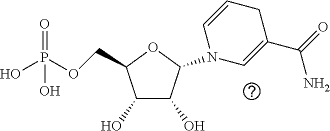 [SMILES: NC(=O)C1=CN([C@H]2O[C@H](COP(=O)(O)O)[C@@H](O)[C@H]2O)C=CC1] |
| I-E (beta) | 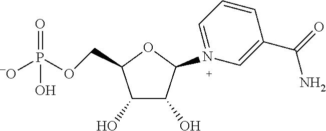 |
| I-F (alpha) | 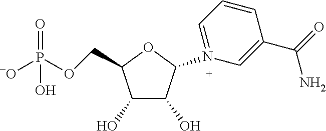 |
| I-G (beta) | 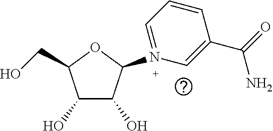 |
| I-H (alpha) | 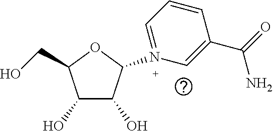 |
| I-I (beta) | 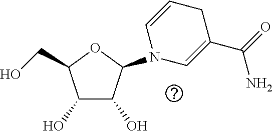 [SMILES: NC(=O)C1=CN([C@@H]2O[C@H](CO)[C@@H](O)[C@H]2O)C=CC1] |
| I-J (alpha) | 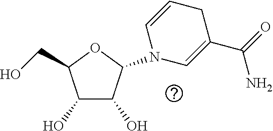 [SMILES: NC(=O)C1=CN([C@H]2O[C@H](CO)[C@@H](O)[C@H]2O)C=CC1] |

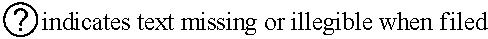

Preferably, the compound having the formula (I) is selected from among: Compound I-A, Compound I-B, Compound I-C, Compound I-D, Compound I-E, Compound I-F, Compound I-G, Compound I-H, Compound I-I, Compound I-J; preferably Compound I-C, Compound I-D or Compound I-F, and combinations thereof. On a more preferred basis, the compound having the formula (I) is selected from among Compound I-B, Compound I-C, Compound I-D, Compound I-F, and combinations thereof.

Derivative Preparation Method for Preparing the Derivatives Having the Formula (Ia)

In particular, the compounds having the formula Ia presented herein may be prepared as described here below from the substrates X-XIII. It is to be understood by the average person skilled in the art that these diagrams are by no means intended to be limiting and that variations thereto in terms of the detail may be made without departing in spirit and scope from the present invention.

According to one embodiment, the invention relates to a compound preparation method for preparing the compound having the formula I described here above.

The method consists first of all in mono-phosphorylating a compound having the formula X, in the presence of phosphoryl chloride in a trialkyl phosphate, in order to obtain the compound phosphorodichloridate XI,

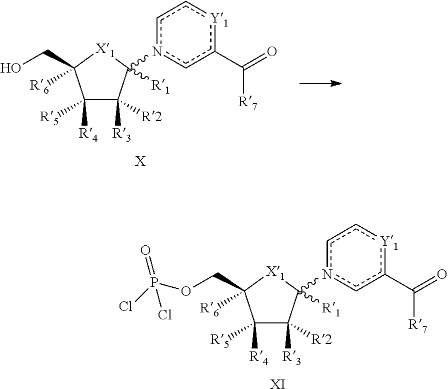

in which X′₁, R′₁, R′₂, R′₃, R′₄, R′₅, R′₆, R′₇, Y′₁, [symbol] and [symbol] are as defined here above.

In a second step, the hydrolysis of the phosphorodichloridate XI obtained in the first step gives the phosphate compound having the formula XII,

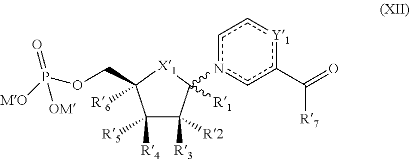

in which X′₁, R′₁, R′₂, R′₃, R′₄, R′₅, R′₆, R′₇, Y′₁, M′, [symbol] and [symbol] are as defined here above.

The phosphate compound having the formula XII obtained in the second step is then reacted with a phosphorodichloridate compound having the formula XIII obtained as described in the first step,

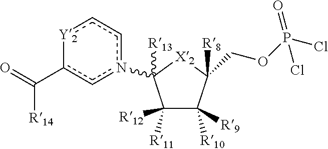

in which X′₂, R′₈, R′₉, R′₁₀, R′₁₁, R′₁₂, R′₁₃, R′₁₄, Y′₂, [symbol] and [symbol] and are as described herein for formula Ia, in order to give the compound having the formula Ia as described herein.

According to one embodiment, the method further comprises a reduction step of reducing the compound having the formula Ia, using various methods known to specialists, in order to give the compound having the formula Ia, where Y′₁ and Y′₂ are identical and each represent CH₂, and where X′₁, X′₂, R′₁, R′₂, R′₃, R′₄, R′₅, R′₆, R′₇, R′₈, R′₉, R′₁₀, R′₁₁, R′₁₂, R′₁₃, R′₁₄, Y′₁, Y′₂, and [symbol] , [symbol] are as described herein for formula Ia.

In one variant, R is a suitable protecting group known to the person skilled in the art. Triarylmethyl and/or silyl groups are examples of suitable protecting groups. Without limitation, some examples of triarylmethyl include trityl, monomethoxytrityl, 4,4′-dimethoxytrityl, and 4,4′,4″-trimethoxytrityl. Without limitation, some examples of silyl groups include trimethylsilyl, tert-butyldimethylsilyl, triisopropylsilyl, tert-butyldiphenylsilyl, tri-iso-propylsilyloxymethyl, and [2-(trimethylsilyl)ethoxy]methyl.

According to one representation, any hydroxy group attached to the pentose ring is protected by a suitable protecting group known to the person skilled in the art.

The selection and exchanging of the protecting groups is well within the scope of knowledge and expertise of the person skilled in the art. Any protecting group may also be removed by methods known in the art, for example, with an acid (for example, an inorganic or organic acid), a base or a fluoride source.

According to one preferred embodiment, the nitrogen compounds having the formula XV are added to the pentose XIV by a coupling reaction in the presence of a Lewis acid in order to give the compound having the formula X-1. Without limitation, some examples of suitable Lewis acids include Trimethylsilyl Trifluoromethanesulfonate (TMSOTf), BF₃.OEt₂, TiCl₄ and FeCl₃.

According to one specific embodiment, the invention relates to a compound preparation method for preparing the compound having the formula VIII,

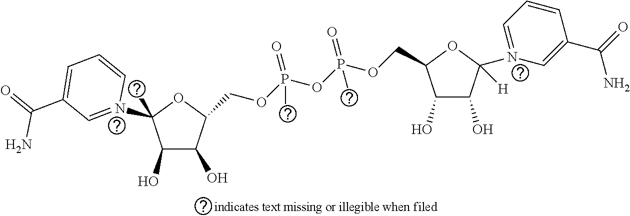

or the pharmaceutically acceptable salts and/or solvates thereof.

In a first step, the nicotinamide having the formula XV is added to the ribose tetraacetate XIV, by a coupling reaction in the presence of a Lewis acid, in order to give the compound having the formula X-1:

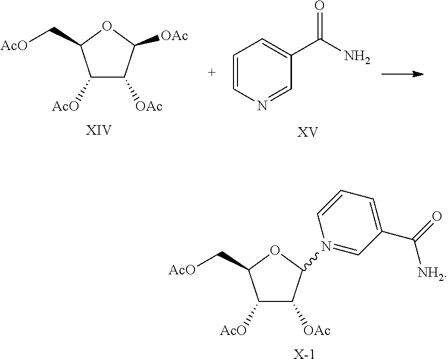

In a second step, an ammoniacal treatment of the compound having the formula X-1 gives the compound having the formula X:

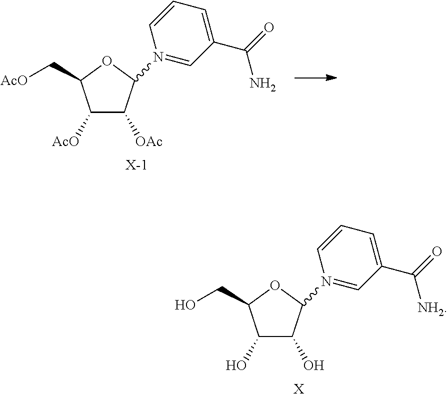

In a third step, the mono-phosphorylation of a compound having the formula X, in the presence of phosphoryl chloride in a trialkyl phosphate, gives the compound phosphorodichloridate XI:

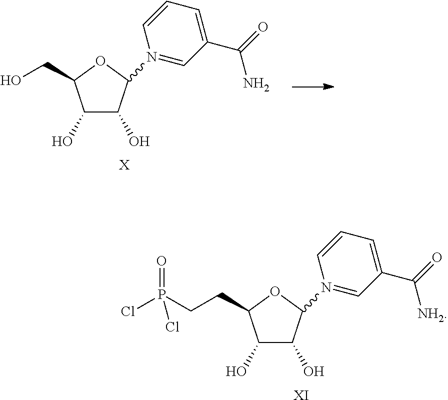

In a fourth step, the phosphorodichloridate compound XI obtained in the third step is partially hydrolysed in order to give the phosphate compound having the formula XII:

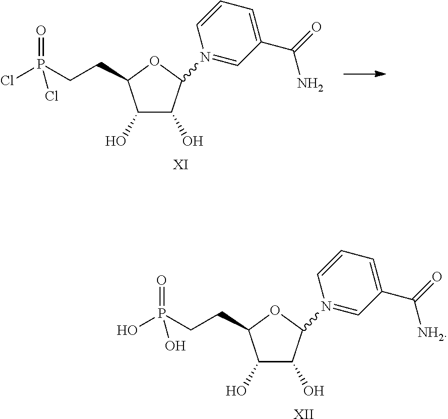

In a fifth step, the phosphate compound having the formula XII obtained in the fourth step is then reacted with the phosphorodichloridate compound having the formula XI obtained as described in the third step, in order to obtain the compound having the formula VIII.

According to another specific implementation embodiment, the invention relates to a compound preparation method for preparing the compound having the formula IX,

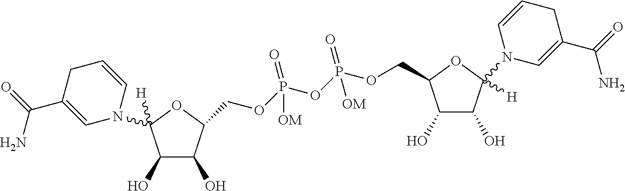

or the pharmaceutically acceptable salts and/or solvates thereof.

According to one variant, the compound having the formula IX is obtained from the compound having the formula VIII, which is synthesised beforehand as described here above.

In this embodiment, the compound having the formula IX is obtained by reducing the compound having the formula VIII, using a suitable reducing agent known to the specialised person skilled in the art, in order to give the compound having the formula IX.

According to one embodiment, the preferred compounds of the invention are the compounds Ia-A to Ia-I of Table 2:

TABLE 2

| Compounds | |
|---|---|
| (Anomers) | Structure |
| Ia-A (beta, beta) | 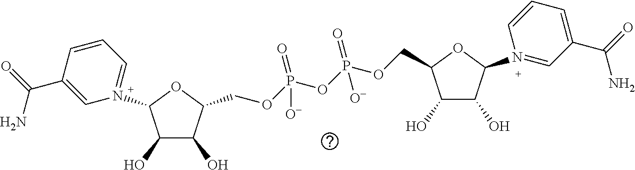 |
| Ia-B (beta, alpha) | 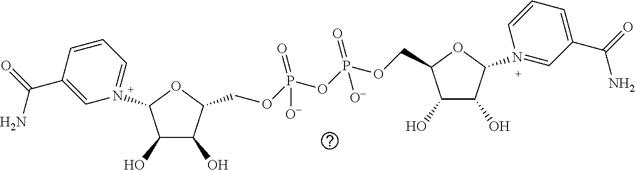 |
| Ia-C (alpha, alpha) | 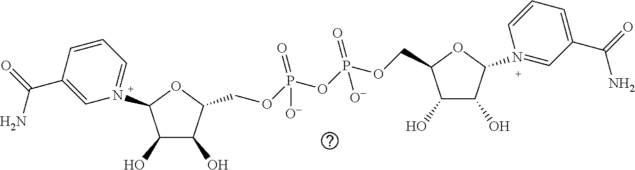 |
| Ia-D (beta, beta) | 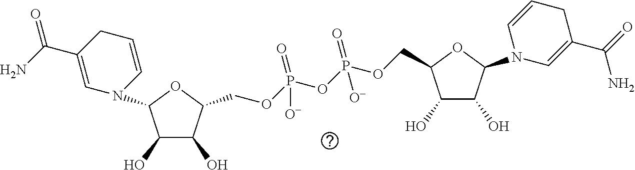 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| Ia-E (beta, alpha) | 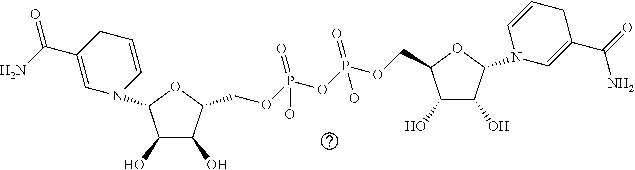 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-F (alpha, alpha) | 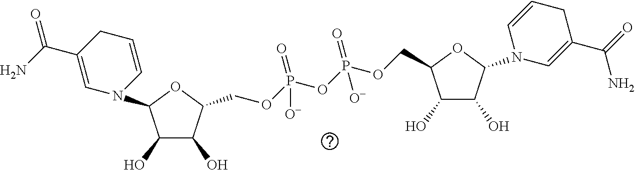 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-G (beta, beta) | 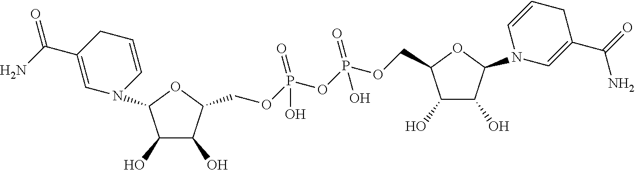 [SMILES: NC(=O)C1=CN([C@@H]2O[C@H](COP(=O)(O)OP(=O)(O)OC[C@H]3O[C@@H](N4C=CCC(C(N)=O)=C4)[C@H](O)[C@@H]3O)[C@@H](O)[C@H]2O)C=CC1] |
| Ia-H (beta, alpha) | 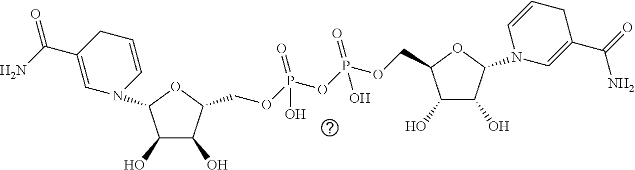 [SMILES: NC(=O)C1=CN([C@@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |
| Ia-I (alpha, alpha) | 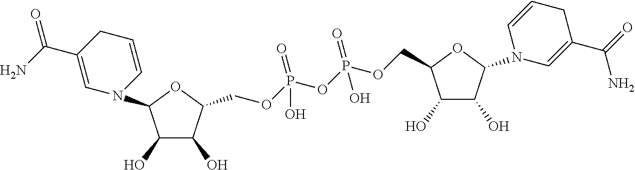 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

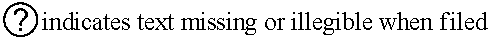

Preferably, the compound having the formula (Ia) is selected from among the compound having the formula Ia-B, the compound having the formula Ia-C, the compound having the formula Ia-E, the compound having the formula Ia-F, the compound having the formula Ia-H, the compound having the formula Ia-I, and the compound having the formula Ia-G as well as combinations thereof.

### FIGURES

FIG. 1 is a graph showing the evolution of the overall pain over the 6 days of application.

FIG. 2 is a graph showing the evolution of the pain with the painful area not being subjected to stress (solicitation) over the 6 days of application.

FIG. 3 is a graph showing the evolution of the pain with the painful area being subjected to stress (solicitation) over the 6 days of application.

FIG. 4 is a graph showing the evolution of the stiffness in the painful area over the 6 days of application.

FIG. 5 is a graph showing the evolution of the difficulty experienced in performing the actions of daily life over the 6 days of application.

### EXAMPLES

In the remainder of this description, the examples provided are intended by way of illustration of the present invention and are in no way intended to limit the scope thereof.

### Example 1

### Synthesis of the Compounds According to the Invention

### Material and Methods

All the reagents were obtained from commercial suppliers and used without any further purification. Thin layer chromatography was carried out on TLC silica gel 60 F254 plastic sheets (0.2 mm layer thickness) from Merck. Purification by column chromatography was carried out on silica gel 60 (70-230 mesh ASTM, Merck). The melting points were determined either on a digital device (Electrothermal IA 8103) and are not corrected, or on a Kofler heating bench of type WME (Wagner & Munz). The ¹H, ¹⁹F, and ¹³C nuclear magnetic resonance (NMR) and infrared (IR) spectra confirmed the structures of all of the compounds. The IR spectra were recorded on a Perkin Elmer Spectrum 100 FT-IR spectrometer; and the NMR spectra were recorded, using CDCl₃, CD₃CN, D₂O or DMSO-d₆ as solvent, on a BRUKER AC 300 or 400 spectrometer at 300 or 400 MHz for the ¹H spectra, 75 or 100 MHz spectra for the ¹³C spectra, and 282 or 377 MHz for the ¹⁹F spectra. The chemical shifts (δ) were expressed in parts per million relative to the signal, indirectly (i) with CHCl₃ (δ 7.27) for ¹H; and (ii) with CDCl₃ (δ 77.2) for ¹³C; and directly (iii) with CFCl₃ (internal standard) (δ 0) for ¹⁹F. The chemical shifts are provided in ppm and the peak multiplicities are denoted as follows: s, singlet; br s, broad singlet; d, doublet; dd, doublet of doublets; ddd, doublet of doublets of doublets; t, triplet; q, quartet; quint, quintet; m, multiplet. High-resolution mass spectra (HRMS) were obtained from the “Service central d'analyse de Solaize” (French National Centre for Scientific Research—Solaize) and were recorded on a Waters spectrometer using electrospray ionisation time-of-flight (ESI-TOF) mass spectrometry. Tetramethylsilane (TMS) having the formula Si(CH₃)₄ is used as reference compound for the NMR spectra.

### Protocol

Step 1—Synthesis of the Compound having the Formula X-1: The compound having the formula XIV (1.0 equiv.) is dissolved in dichloromethane. The nicotinamide having the formula XV (1.50 equiv.) and the TMSOTf (1.55 equiv.) are added at ambient temperature. The reaction mixture is heated under reflux and stirred until completion of the reaction. The mixture is cooled to ambient temperature and filtered. The filtrate is concentrated to dryness so as to give crude NR (nicotinamide riboside) tetraacetate having the formula X-1.

Step 2—Synthesis of the Compound Having the Formula X: The crude NR tetraacetate having the formula X-1 is dissolved in methanol and cooled to −10° C. This is followed by addition of 4.6 M ammonia in methanol (3.0 equivalents) at −10° C. and the mixture is stirred at this temperature until completion of the reaction. Dowex HCR (H*) is added until a pH of 6-7 is attained. The reaction mixture is heated to 0° C. and filtered. The resin is washed with a mixture of methanol and acetonitrile. The filtrate is concentrated until it becomes dry. The residue is dissolved in acetonitrile and concentrated to solid content dryness. The residue is dissolved in acetonitrile so as to give a solution of crude nicotinamide riboside triflate having the formula X.

Step 3—Synthesis of the Compound having the Formula XI: The solution of crude NR nicotinamide riboside triflate in acetonitrile is diluted with trimethyl phosphate (10.0 equivalents). The acetonitrile is distilled under vacuum and the mixture is cooled to −10° C. Phosphorus oxychloride (4.0 equiv.) is added at −10° C. and the mixture is stirred at −10° C. until completion of the reaction.

Step 4 and Step 5: Synthesis of the Compound having the Formula Ia-A: The mixture is hydrolysed by adding a 50/50 mixture of acetonitrile and water, followed by the addition of methyl tert-butyl ether (or tert-butyl methyl ether). The mixture is filtered and the solid is dissolved in water. The aqueous solution is neutralised by adding sodium bicarbonate and extracted with dichloromethane. The aqueous layer is concentrated to dryness so as to give a crude mixture of NMN (Compound I-A) and the compound having the formula Ia-A.

Isolation of the Compound having the Formula Ia-A (β,βdiNMN): The NMN and the compound having the formula Ia-A are separated by purification on Dowex 50wx8 with elution of water. The fractions containing the compound having the formula Ia-A are concentrated to solid content dryness. The residue is purified by column chromatography on silica gel (isopropanol/water gradient). The pure fractions are combined and concentrated. The residue is lyophilised so as to give the Compound I-a-A in the form of a beige solid.

³¹P NMR: δ (ppm, reference 85% H₃PO₄: 0 ppm in D₂O)=−11.72; ¹H NMR: δ (ppm, reference TMS: 0 ppm in D₂O)=4.20 (ddd, J_{H-H}=11.9, 3.5, 2.4 Hz, 2H), 4.35 (ddd, J_{H-H}=11.9, 3.9, 2.2 Hz, 2H), 4.43 (dd, J_{H-H}=5.0, 2.6 Hz, 2H), 4.53 (t, J_{H-H}=5.0 Hz, 2H), 4.59 (m, 2H), 6.16 (d, J_{H-H}=5.4 Hz, 2H), 8.26 (dd, J_{H-H}=8.1, 6.3 Hz, 2H), 8.93 (d, J_{H-H}=8.1 Hz, 2H), 9.25 (d, J_{H-H}=6.2 Hz, 2H), 9.41 (s, 2H); ¹³C NMR: δ (ppm, reference TMS: 0 ppm in D₂O)=64.84 (CH₂), 70.73 (CH), 77.52 (CH), 87.11 (CH), 99.88 (CH), 128.65 (CH), 133.89 (Cq), 139.84 (CH), 142.54 (CH), 146.04 (CH), 165.64 (Cq); MS (ES+): m/z=122.8 [Mnicotinamide+H]+, 650.8 [M+H]+.

Synthesis of the Compound Having the Formula Ia-B (α,β di-NMN)

Phosphorus oxychloride (3.0 eq.) is added to trimethylphosphate (20.0 eq.) at −5° C. β-NR chloride (1.0 eq.) is added in portions at −5° C. and the reaction mixture is stirred overnight at −5° C. Morpholine (3.0 eq.) is added dropwise at −10/0° C. and the mixture is stirred for 2-3 hrs. α-NMN (1.0 eq.) is then added in portions at −5° C. and the reaction mixture is stirred at −5° C. overnight. The hydrolysis is carried out by dropwise addition of water (5 vol.) at −10/0° C. and the mixture is stirred until complete homogenisation at 10-15° C. The reaction mixture is then extracted with dichloromethane (6*10 vol.) and the aqueous phase is neutralised by elution through the formate resin Purolite A600E (theoretical quantity to neutralise the HCl originating from POCl₃). The eluate is then concentrated in vacuo at 45/50° C. in order to give the crude containing the compound having the formula Ia-B. The water elution with the H+ resin Dowex 50wx8 100-200 mesh makes it possible to remove certain impurities. The fractions containing the Compound I-B are combined and concentrated in vacuo at 45-50° C. The crude is then purified by preparative chromatography on Luna Polar RP 10 μm stationary phase with elution with an aqueous solution of 10 mM NaH2PO4. The pure fractions are combined and eluted with water on the resin Purolite C100EH H+ (quantity necessary to completely exchange Na+ by H+), then eluted on the resin Purolite A600E acetate (quantity necessary to completely exchange H₂PO₄— by acetate). The eluate is concentrated in vacuo and the residue is lyophilised in order to give the Compound Ia-B in the form of a white solid.

³¹P NMR: δ (ppm, reference 85% H₃PO₄: 0 ppm in D₂O)=−11.87, −11.69, −11.46, −11.29; ¹H NMR: δ (ppm, reference TMS: 0 ppm in D₂O)=4.10 (ddd, J=11.1, 6.1, 3.1 Hz, 1H), 4.15-4.25 (m, 2H), 4.36 (ddd, J=12.2, 4.4, 2.4 Hz, 1H), 4.40 (dd, J=4.9, 2.4 Hz, 1H), 4.44 (dd, J=5.0, 2.7 Hz, 1H), 4.53 (t, J =5.0 Hz, 1H), 4.5 (m, 1H), 4.85 (m, 1H), 4.92 (t, J=5.3 Hz, 1H), 6.15 (d, J=5.5 Hz, 1H), 6.51 (d, J =5.7 Hz, 1H), 8.14 (dd, J=8.0, 6.3 Hz, 1H), 8.26 (dd, J=8.1, 6.3 Hz, 1H), 8.88 (d, J=8.1 Hz, 1H), 8.92 (d, J=8.1 Hz, 1H), 9.02 (d, J=6.3 Hz, 1H), 9.24 (s, 1H), 9.26 (d, J=6.4 Hz, 1H), 9.40 (s, 1H); ¹³C NMR: δ (ppm, reference TMS: 0 ppm in D₂O)=64.83, 64.87 (CH2), 65.30, 65.35 (CH2), 70.65 (CH), 70.74 (CH), 71.92 (CH), 77.51 (CH), 87.03, 87.10 (CH), 87.19, 87.26 (CH), 96.57 (CH), 99.83 (CH), 126.89 (CH), 128.54 (CH), 132.44 (Cq), 133.81 (Cq), 139.85 (CH), 140.92 (CH), 142.50 (CH), 143.49 (CH), 145.06 (CH), 145.97 (CH), 165.64 (Cq), 165.88 (Cq); MS (ES+): m/z=122.8 [Mnicotinamide+H]+, 650.9 [M+H]+.

Synthesis of the Compound Having the Formula Ia-C (α, α di-NMN)

Phosphorus oxychloride (3.0 eq.) is added to trimethylphosphate (20.0 eq.) at −5° C. α-NR chloride (1.0 eq.) is added portionwise at −5° C. and the reaction mixture is stirred overnight at −5° C. Morpholine (3.0 eq.) is added dropwise at −10/0° C. and the mixture is stirred for 2-3 hrs. α-NMN (1.0 eq.) is then added in portions at −5° C. and the reaction mixture is stirred at −5° C. overnight. The hydrolysis is carried out by dropwise addition of water (5 vol.) at −10/0° C. and the mixture is stirred until complete homogenisation at 10-15° C. The reaction mixture is then extracted with dichloromethane (6*10 vol.) and the aqueous phase is neutralised by elution through the formate resin Purolite A600E (theoretical quantity to neutralise the HCl originating from POCl₃). The eluate is then concentrated in vacuo at 45/50° C. to give the crude containing the compound having the formula Ia-C. The water elution with H+ resin Dowex 50wx8 100-200 mesh makes it possible to remove certain impurities. The fractions containing the Compound I-C are combined and concentrated in vacuo at 45-50° C. The crude is then purified by preparative chromatography on Luna Polar RP 10 μm stationary phase with elution with an aqueous solution of 10 mM NaH₂PO₄. The pure fractions are combined and eluted with water on resin Purolite C100EH H+ (quantity necessary to completely exchange Na+ by H+), then eluted on on the resin Purolite A600E acetate (quantity necessary to completely exchange H₂PO₄— by acetate). The eluate is concentrated in vacuo and the residue is freeze-dried in order to give the Compound Ia-C in the form of a white solid.

³¹P NMR: δ (ppm, reference 85% H₃PO₄: 0 ppm in D₂O)=−11.40; ¹ H NMR: δ (ppm, reference TMS: 0 ppm in D₂O)=4.14 (ddd, J=11.4, 3.4, 2.8 Hz, 2H), 4.23 (ddd, J=11.6, 3.3, 2.8 Hz, 2H), 4.44 (dd, J=4.8, 2.3 Hz, 2H), 4.88 (m, 2H), 4.96 (t, J=5.3 Hz, 2H), 6.54 (d , J=5.7 Hz, 2H), 8.15 (dd, J=8.1, 6.2 Hz, 2H), 8.89 (d, J=8.1 Hz, 2H), 9.05 (d, J=6.3 Hz, 2H), 9.26 (s, 2H); ¹³C NMR: δ (ppm, reference TMS: 0 ppm in D₂O)=65.37 (CH2), 70.70 (CH), 71.95 (CH), 87.30 (CH), 96.62 (CH), 126.91 (CH), 132.45 (Cq), 140.94 (CH), 143.52 (CH), 145.07 (CH), 165.90 (Cq); MS (ES+): m/z=122.7 [Mnicotinamide+H]+, 650.8 [M+H]+.

### Example 2

### Study of the Efficacy of Compound I-A (NMN Beta)

A satisfaction study was carried out on a group of 12 volunteers, aged 34.5±11.1 years, consisting of seven female and five male subjects. The main objective of this study was to evaluate the level of satisfaction of subjects with regard to the evolution of their muscle and/or tendon pain during the morning and/or evening application of a gel containing 5% by weight of NMN.

The average BMI of the participants was 24.1±3.7 kg/m². Specifically, six participants were of normal weight, four participants were overweight, and two participants were obese. None of these patients presented with a chronic pathology such as osteoarthritis, a tumour, a neurological or genetic disease, an inflammatory pathology altering their cartilage, muscles, tendons, ligaments or bones, or requiring surgery.

The duration of the existence of the pain in the knee at the time of the study was on average 2.1±0.8 days. These pains had occurred mainly following the practise of a physical activity (83.3%) or an activity such as gardening (8.3%). One person presented with spontaneous pain. The 11 other volunteers had pain following the practise of a sport or a physical activity.

The pains were mainly localised in the thighs (66.7%) and the shoulder (41.7%). However, participants also experienced muscle and/or tendon pain in the leg, neck, arm, hip, wrist, hand, and elbow.

A composition in the form of an oil-in-water emulsion comprising 5% NMN was formulated as follows, the ingredients being designated by their INCI (International Nomenclature of Cosmetic Ingredients) name: Aqua, Paraffinum liquidum, Cetyl alcohol, Glyceryl stearate, Benzyl PCA, Ceteareth-20, Ceteareth-12, Cetyl Palmitate, Cocoglycerides, Cetearyl alcohol, Sodium Hydroxide, NMN.

The mass percentages are calculated by relating the mass of the ingredient to the total mass of the composition, then by multiplying by 100.

The study took place over 6 days. At baseline, ie upon inclusion (D0), the selected subjects provide their demographic characteristics (age, weight, height), indicate the period of persistence and intensity of the pain on a Visual Analogue Scale. More precisely, the subjects rate their pain actually experienced on a scale from 0% (no pain at all) to 100% (most extreme pain). The difference in the measurements of the pain intensity felt from one day to the next provides information on the pain relief experienced by the patient.

Over the subsequent 5 day period, each evening the subjects fill in the Visual Analogue Scale of pain, ensuring any discomfort or problems arising or the taking of analgesics are reported therein.

On day 6, the volunteers complete the WOMAC questionnaire, the Lequesne questionnaire, the Visual Analogue Scale (VAS) of pain, indicating the perceived improvement in lumbalgia pain relief as measured by the PGI-I index (abbreviation for “Patient Global Improvement Impression”), satisfaction with regard to evolution of the muscle and/or tendon pain on a Likert scale as well as the ease of application and penetration of the gel, assessment of the texture and odour of the gel, likelihood of re-use thereof in the event of recurrence of a similar pain, and recommendation of use to third parties presenting with pain of similar nature. The PGI-I index is an index that serves as means for evaluating the response to a treatment. The Likert scale is a psychometric tool used for measuring an attitude in individuals, which consists of one or more statements for which the individual responding expresses their degree of agreement or disagreement. The statistical analyses were carried out by means of an ANOVA test, also called analysis of variance test, and a Student's t test.

Treatment compliance was 89.2% on average during the study. About 75% of the subjects applied the composition according to the invention twice a day, versus 25% who applied the composition only once a day.

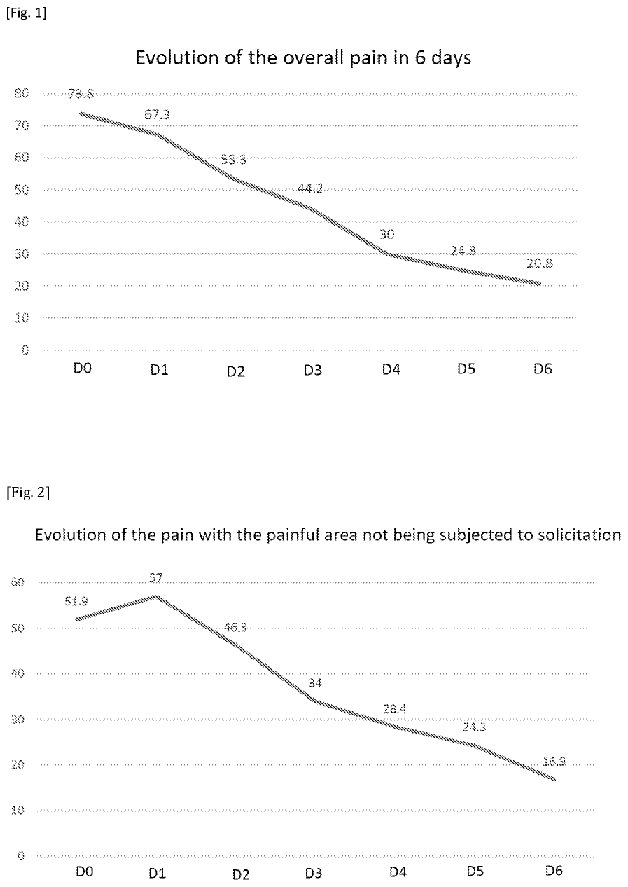

With regard to the evolution of the overall pain as measured by the patients on the VAS scale, it may be seen in FIG. 1 which summarises these data that the pain decreased steadily in a constant manner over the five days of application of the composition comprising NMN according to the invention, dropping from an average of 73.8±5.7 at D0 to an average of 20.8±26.2 at D6. In particular, the composition comprising NMN according to the invention served to enable an overall pain reduction of 72.4%±34.2% (p<0.0001 Anova, p<0.0001 Student). As to the mean time period for achieving a 50% reduction in pain as compared to D0, this was obtained after 3.8±1.4 days. The composition comprising NMN according to the invention therefore served to enable a significant and rapid reduction in the muscle and/or tendon pain experienced by the subjects in the study.

According to FIG. 2, the pain with the painful area not being subjected to stress (solicitation) decreased steadily in a constant manner during the five days of application of the gel, with the exception of day 1, declining from 51.9±19.2 at baseline to 16.9±22.9 which is a significant reduction of 75.2%±32.3% (p<0.0001 Anova, p<0.0001 Student).

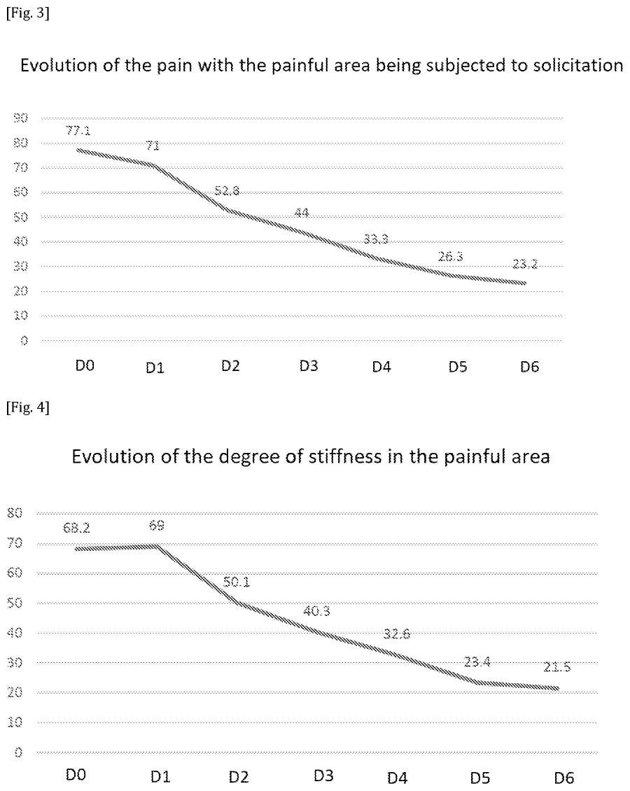

According to FIG. 3, the pain with the painful area being subjected to stress (solicitation) decreased steadily in a constant manner over the five days of application of the gel, declining from 77.1±6.7 at baseline to 23.2±27.4, which is a significant reduction of 68.7%±37.7%. (p<0.0001 Nova, p<0.0001 Student).

The composition comprising NMN according to the invention therefore makes it possible to reduce the muscle and/or tendon pain experienced by the subjects, even without resting the muscle group concerned.

The stiffness in the painful area was also measured by means of the VAS scale. According to FIG. 4, the severity of the stiffness in the painful area decreased steadily in a constant manner (except on day 1) during the five days of application of the gel declining from 68.2±14.3 at baseline to 21.5±26.5 which is a significant reduction of 66.6%±39.9% (p<0.0001 Anova, p<0.0001 Student). The composition comprising NMN according to the invention therefore makes it possible to reduce muscle and/or tendon stiffness.

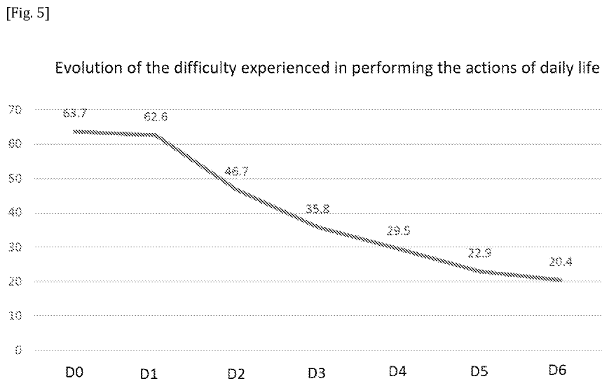

The subjects of the study were also asked about the difficulty experienced in performing the actions of daily life, measured by the VAS scale. According to FIG. 5, the difficulty experienced in performing the actions of daily life decreased steadily in a constant manner over the five days of application of the gel, declining from 63.7±15.5 at baseline to 20.4±25.6, which is a significant reduction of 69.6% ±35.4% (p<0.0001 Anova, p<0.0001 Student). The composition comprising NMN according to the invention therefore makes it possible to improve muscle and/or tendon function.

At the end of the study, 10 subjects experienced significant improvement. About 91.7% of the volunteers were satisfied with the evolution of their muscle and/or tendon pain, of which 41.7% were very satisfied.

During the study, no analgesics or non-steroidal anti-inflammatory drugs were taken by any of the subjects, whether on account of muscle and/or tendon pain experienced following physical or sporting activity, or on account of another illness. NMN and the compositions that comprise the same therefore make it possible to avoid having to resort to conventional treatments.

From an organoleptic standpoint, all the patient responses confirmed that the gel: was easy to apply (75%—very easy); penetrated easily into the skin (83.4%—very easily); had a pleasant texture (66.7%—very pleasant); and a pleasant smell. With the exception of one subject who found that there had not been sufficient improvement in the pain, all the other study participants confirmed their likelihood of using the gel again in the event of recurrence of muscle, ligament and/or tendon pain.

Finally, the subjects experienced neither any adverse side effects following the use of the composition according to the invention nor developed any allergies.

NMN, a pharmaceutically acceptable derivative thereof, or a pharmaceutically acceptable salt thereof, as well as compositions that comprise the same are therefore effective in reducing muscle, ligament and/or tendon pain induced by physical or sports activity.

Indeed, regardless of the pain measurement scale used, a significant reduction in the pain induced by physical activity was measured in study participants. Furthermore, the composition comprising NMN according to the invention made it possible to avoid taking analgesics and nonsteroidal anti-inflammatory drugs conventionally used to relieve muscle and tendon pain. The inventors have therefore demonstrated that the compositions according to the invention and the NMN, as well as the pharmaceutically acceptable salts and derivatives thereof are effective for relieving pain and for avoiding the need to use conventional therapies. Thus, by reducing the use of conventional therapies, the invention also makes it possible to reduce the adverse side effects thereof.

Although the subjects participating in the invention-related study presented with only muscle, tendon or musculotendinous pain and no ligament pain, the use of NMN and of compositions that comprise the same according to the invention provides the means to obtain the same effects on ligament pain, whether occurring individually or combined with muscle or tendon pain. The results of the present study are also applicable to muscle groups other than the shoulder, thigh, leg, neck, arm, hip, wrist, hand and elbow.

NMN, a pharmaceutically acceptable derivative thereof, or a pharmaceutically acceptable salt thereof, as well as the compositions that comprise the same may therefore be used successfully for treating or preventing other types of muscle, ligament or tendon pains or combinations thereof. Furthermore, NMN, a pharmaceutically acceptable derivative thereof, or a pharmaceutically acceptable salt thereof, as well as the compositions that comprise the same, make it possible to reduce the use of conventional therapies for treating muscle, ligament and/or tendon pain.

1. Nicotinamide mononucleotide (NMN), a pharmaceutically acceptable derivative thereof, or a pharmaceutically acceptable salt thereof, for use thereof in the prevention and/or treatment of a muscle, ligament, or tendon pain or combinations thereof, induced by physical activity.

2. Nicotinamide mononucleotide (NMN), a pharmaceutically acceptable derivative thereof, or a pharmaceutically acceptable salt thereof, for use thereof according to claim 1 in which the pharmaceutically acceptable derivative of NMN is dihydronicotinamide mononucleotide (NMN-H), alpha-NMN, a compound having the formula (I): 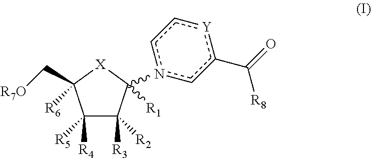
or one of the pharmaceutically acceptable: stereoisomers, salts, hydrates, solvates, or crystals thereof, in which:
X is selected from among O, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄ and R₅ are selected independently of one another, from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from a proteinogenic amino acid;
R₆ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R⁷ is selected from among H, P(O)R₉R₁₀, and P(S)R₉R₁₀ and 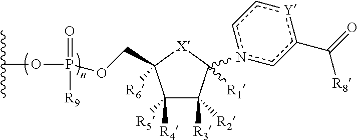
where n is an integer selected from 1 or 3; in which
R₉ and R₁₀ are selected independently of one another, from among OH, OR₁₁, NHR₁₃, NR₁₃R₁₄, a (C₁-C₈) alkyl, a (C₂-C₈) alkenyl, a (C₂-C₈)alkynyl, a (C₃-C₁₀) cycloalkyl, a (C₅-C₁₂) aryl, (C₁-C₈)alkyl aryl, (C₁-C₈) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —(CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)ₙC(O)O(C₁-C₁₅)alkyl, and —(CH₂)ₙC(O)O(C₁-C₁₅)alkyl aryl; wherein n is an integer selected from 1 to 8; P(O)(OH)OP(O)(OH)₂; halogen, nitro, cyano, (C₁-C₆) alkoxy, (C₁-C₆) haloalkoxy, —N(R₁₁ₐ)₂, C₁-C₆ acylamino, —COR_{11b}, —OCOR_{11b}; NHSO₂(C₁-C₆ alkyl), —SO₂N(R₁₁ₐ)₂ SO₂; wherein each of R₁₁ₐ is independently selected from H and a (C₁-C₆) alkyl, and R_{11b} is independently selected from OH, C₁-C₆ alkoxy, NH₂, NH(C₁-C₆ alkyl) or N(C₁-C₆ alkyl)2;
R₁₂ is selected from among H, (C₁-C₁₀) alkyl, (C₂-C₈) alkenyl, (C₂-C₈) alkynyl, (C₁-C₁₀) haloalkyl, (C₃-C₁₀) cycloalkyl, (C₃-C₁₀) heterocycloalkyl, (C₅-C₁₈) aryl, (C₁-C₄) alkylaryl, and (C₅-C₁₂) heteroaryl; wherein the said aryl or heteroaryl groups are optionally substituted with one or two groups selected from among halogen, trifluoromethyl, (C₁-C₆) alkyl, (C₁-C₆)alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl, —(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among hydroxyl, (C₁-C₁₀) alkyl, (C₁-C₆) alkoxy, a halogen, a nitro, and a cyano; or
R₉ and R₁₀ form, together with the phosphorus atoms to which they are attached, a 6-membered ring in which —R₉—R₁₀— represents —CH₂—CH₂—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or
R₉ and R₁₀ form, together with the phosphorus atoms to which they are attached, a 6-membered ring in which —R₉—R₁₀— represents —O—CH₂—CH₂—CHR—O—; wherein R is selected from among H, a (C₅-C₆) aryl group, and (C₅-C₆) heteroaryl, wherein the said aryl or heteroaryl groups are optionally substituted by a halogen, trifluoromethyl, a (C₁-C₆) alkyl, a (C₁-C₆) alkoxy, and cyano;
R₈ is selected from among H, OR, NHR₁₃, NR₁₃R₁₄, NH—NHR₁₃, SH, CN, N₃, and halogen;
wherein R₁₃ and R₁₄ are selected independently of one another, from among H, (C₁-C₈) alkyl, (C₁-C₈) alkyl aryl, and —CR_{B}R_{C}—C(O) —OR_{D} in which R_{B} and R_{C} are independently a hydrogen atom, a (C₁-C₆) alkyl, a (C₁-C₆) alkoxy, benzyl, indolyl, or imidazolyl; where the (C₁-C₆) alkyl and the (C₁-C₆) alkoxy may be optionally and independently of one another substituted by one or more of the halogen, amino, amido, guanidyl, hydroxyl, thiol, or carboxyl groups, and the benzyl group is optionally substituted by one or more halogen or hydroxyl groups; or R_{B} and R_{C} form, together with the carbon atom to which they are attached, a C₃-C₆ cycloalkyl group optionally substituted by one or more halogens, amino, amido, guanidyl, hydroxyl, thiol, and carboxyl; and R_{D} is a hydrogen, a (C₁-C₆) alkyl, a (C₂-C₆) alkenyl, a (C₂-C₆) alkynyl, or a (C₃-C₆) cycloalkyl;
Y is selected from among CH, CH₂, C(CH₃)₂ and CCH₃; [symbol]
represents a single or a double bond along Y; and [symbol]
represents the alpha or beta anomer depending on the position of R₁;
or
a compound having the formula (Ia): 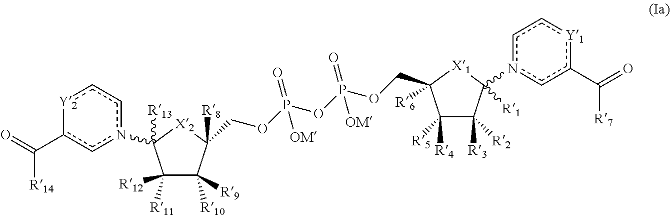
or one of the: stereoisomers, salts, hydrates, solvates, or crystals thereof, in which:
X′₁ and X′₂ are independently selected from among O, CH₂, S, Se, CHF, CF₂, and C═CH₂;
R′₁ and R′13 are independently selected from among H, azido, cyano, a C1-C8 alkyl, a C1-C8 thio-alkyl, a C1-C8 heteroalkyl, and OR, wherein R is selected from H and a C1-C8 alkyl;
R′₂, R′₃, R′₄, R′₅, R′₉, R′₁₀, R′₁₁, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH2 group; wherein R_{AA} is a side chain selected from a proteinogenic amino acid;
R′₆ and R′₈ are independently selected from among H, an azido, a cyano, a C₁-C₈ alkyl and OR, wherein R is selected from H and a C₁-C₈ alkyl;
R′₇ and R′₁₄ are independently selected from among H, OR, NHR, NRR′, NH—NHR, SH, CN, N₃ and a halogen; wherein R and R′ are independently selected from H and a (C₁-C₈) alkyl aryl;
Y′1 and Y′2 are independently selected from among CH, CH₂, C(CH₃)₂, or CCH₃;
M′ is selected from H or a suitable counter ion; [symbol]
represents a single or double bond depending on Y′₁ and Y′₂; and [symbol]
represents an alpha or beta anomer depending on the position of R′₁ and R′₁₃;
and combinations thereof, for use thereof via topical administration in the prevention and/or treatment of a back pain.

3. Nicotinamide mononucleotide (NMN), a pharmaceutically acceptable derivative thereof, or a pharmaceutically acceptable salt thereof, for use thereof according to claim 2 in which the pharmaceutically acceptable derivative of NMN is selected from among:
Compound I-B, Compound I-C, Compound I-D, Compound I-E, Compound I-F, Compound I-G, Compound I-H, Compound I-I, Compound I-J, preferably Compound I-B, Compound I-C, Compound I-D, Compound I-F, and combinations thereof from Table 1.

4. Nicotinamide mononucleotide (NMN), a pharmaceutically acceptable derivative thereof, or a pharmaceutically acceptable salt thereof, for use thereof according to claim 2 is selected from among the compounds Ia-A to Ia-I, preferably from among the compound having the formula Ia-B, the compound having the formula Ia-C, the compound having the formula Ia-E, the compound having the formula Ia-F, the compound having the formula Ia-H, the compound having the formula Ia-I and the compound having the formula Ia-G of Table 2.

5. Nicotinamide mononucleotide (NMN), a pharmaceutically acceptable derivative thereof, or a pharmaceutically acceptable salt thereof, for use thereof according to claim 1, intended to be administered topically.

6. Nicotinamide mononucleotide (NMN), a pharmaceutically acceptable derivative thereof, or a pharmaceutically acceptable salt thereof, for use thereof according to claim 1 wherein the physical activity is the practise of a sport.

7. Nicotinamide mononucleotide (NMN), a pharmaceutically acceptable derivative thereof, or a pharmaceutically acceptable salt thereof, for use thereof in combination with at least one other therapeutic agent.

8. Nicotinamide mononucleotide (NMN), a pharmaceutically acceptable derivative thereof, or a pharmaceutically acceptable salt thereof, for use thereof according to claim 1 in which the muscle pain is selected from among: soreness, contracture, cramping, elongation, muscle contusion, muscle tearing, partial or complete rupture of muscle fibres, or combinations thereof the ligament pain is selected from a sprain, a partial or complete tearing of the ligament or combinations thereof and the tendon pain is selected from tendonitis, tenosynovitis, bursitis or combinations thereof.

9. A composition comprising nicotinamide mononucleotide (NMN), a pharmaceutically acceptable derivative thereof, or a pharmaceutically acceptable salt thereof, and at least one pharmaceutically acceptable excipient, for use thereof in the prevention and/or treatment of muscle, ligament, or tendon pain or combinations thereof, induced by physical activity.

10. The composition according to claim 9 intended to be administered via the topical route.

11. The composition according to claim 9 comprising nicotinamide mononucleotide (NMN), a pharmaceutically acceptable derivative thereof or a pharmaceutically acceptable salt thereof, in an amount between 0.05% and 15% by weight, preferably between 1% and 10% by weight, more preferably between 3% and 5% by weight based on the total weight of the composition.

12. The composition according to claim 9 further comprising at least one additional therapeutic agent.

13. The composition according to claim 9, in which the pharmaceutically acceptable derivative is selected from among dihydronicotinamide mononucleotide (NMN-H), alpha-NMN, a compound having the formula (I): 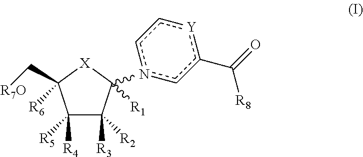
or one of the pharmaceutically acceptable: stereoisomers, salts, hydrates, solvates, or crystals thereof, in which:
X is selected from among O, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄ and R₅ are selected independently of one another, from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from a proteinogenic amino acid;
R₆ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₇ is selected from among H, P(O)R₉R₁₀, and P(S)R₉R₁₀ and 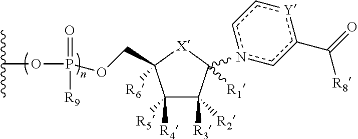
where n is an integer selected from 1 or 3; in which
R₉ and R₁₀ are selected independently of one another, from among OH, OR₁₁, NHR₁₃, NR₁₃R₁₄, a (C₁-C₈) alkyl, a (C₂-C₈) alkenyl, a (C₂-C₈)alkynyl, a (C₃-C₁₀) cycloalkyl, a (C₅-C₁₂) aryl, (C₁-C₈)alkyl aryl, (C₁-C₈) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —(CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)ₙC(O)O(C₁-C₁₅)alkyl, and —(CH₂)ₙC(O)O(C₁-C₁₅)alkyl aryl; wherein n is an integer selected from 1 to 8; P(O)(OH)OP(O)(OH)₂; halogen, nitro, cyano, C₁-C₆ alkoxy, C₁-C₆ haloalkoxy, —N(R₁₁ₐ)₂, C₁-C₆ acylamino, —COR_{11b}, —O COR_{11b}; NHSO₂(C₁-C₆ alkyl), —SO₂N(R₁₁ₐ)₂ SO₂; wherein each of R₁₁ₐ is independently selected from H and a (C₁-C₆) alkyl, and R_{11b} is independently selected from OH, C₁-C₆ alkoxy, NH₂, NH(C₁-C₆ alkyl) or N(C₁-C₆ alkyl)₂;
R₁₂ is selected from among H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl;
wherein the said aryl or heteroaryl groups are optionally substituted with one or two groups selected from among halogen, trifluoromethyl, C₁-C₆ alkyl, C₁-C₆ alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl, —(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among hydroxyl, (C₁-C₁₀) alkyl, (C₁-C₆) alkoxy, a halogen, a nitro, and a cyano; or
R₉ and R₁₀ form, together with the phosphorus atoms to which they are attached, a 6-membered ring in which —R₉—R₁₀— represents —CH₂—CH₂—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or
R₉ and R₁₀ form, together with the phosphorus atoms to which they are attached, a 6-membered ring in which —R₉—R₁₀— represents —O—CH₂—CH₂—CHR—O—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano;
R₈ is selected from among H, OR, NHR₁₃, NR₁₃R₁₄, NH—NHR₁₃, SH, CN, N₃, and halogen;
wherein R₁₃ and R₁₄ are selected independently of one another, from among H, (C₁-C₈) alkyl, (C₁-C₈) alkyl aryl, and —CR_{B}R_{C}—C(O) —OR_{D} in which R_{B} and R_{C} are independently a hydrogen atom, a (C₁-C₆) alkyl, a (C₁-C₆) alkoxy, benzyl, indolyl, or imidazolyl; where the (C₁-C₆) alkyl and the (C₁-C₆) alkoxy may be optionally and independently of one another substituted by one or more of the halogen, amino, amido, guanidyl, hydroxyl, thiol, or carboxyl groups, and the benzyl group is optionally substituted by one or more halogen or hydroxyl groups; or R_{B} and R_{C} form, together with the carbon atom to which they are attached, a C₃-C₆ cycloalkyl group optionally substituted by one or more halogens, amino, amido, guanidyl, hydroxyl, thiol, and carboxyl; and R_{D} is a hydrogen, a (C₁-C₆) alkyl, a (C₂-C₆) alkenyl, a (C₂-C₆) alkynyl, or a (C₃-C₆) cycloalkyl;
Y is selected from among CH, CH₂, C(CH₃)₂ and CCH₃; [symbol]
represents a single or a double bond along Y; and [symbol]
represents the alpha or beta anomer depending on the position of R₁;
or
a compound having the formula (Ia): 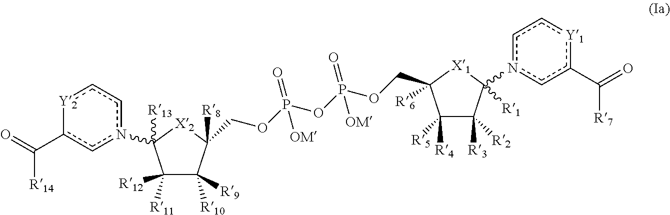
or one of the stereoisomers, salts, hydrates, solvates, or crystals thereof, in which:
X′₁ and X′₂ are independently selected from among O, CH₂, S, Se, CHF, CF₂, and C═CH₂;
R′₁ and R′13 are independently selected from among H, azido, cyano, a C1-C8 alkyl, a C1-C8 thio-alkyl, a C1-C8 heteroalkyl, and OR, wherein R is selected from H and a C1-C8 alkyl;
R′₂, R′₃, R′₄, R′₅, R′₉, R′₁₀, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH2 group; wherein R_{AA} is a side chain selected from a proteinogenic amino acid;
R′₆ and R′₈ are independently selected from among H, an azido, a cyano, a C₁-C₈ alkyl and OR, wherein R is selected from H and a C₁-C₈ alkyl;
R′₇ and R′₁₄ are independently selected from among H, OR, NHR, NRR′, NH—NHR, SH, CN, N₃ and a halogen; wherein R and R′ are independently selected from H and a (C₁-C₈) alkyl aryl;
Y′1 and Y′2 are independently selected from among CH, CH₂, C(CH₃)₂, or CCH₃;
M′ is selected from H or a suitable counter ion; [symbol]
represents a single or double bond depending on Y′₁ and Y′₂; and [symbol]
represents an alpha or beta anomer depending on the position of R′₁ and R′₁₃;
and combinations thereof, for use thereof via topical administration in the prevention and/or treatment of a muscle, ligament, tendon or their combination, induced by physical activity.

14. The composition according to claim 13 wherein the pharmaceutically acceptable derivative of NMN is selected from compound I-B, compound I-C, compound I-D, compound I-E, compound I-F, compound I-G, compound I-H, compound I-I, compound I-J, preferably compound IB , compound IC, compound ID, compound IF from Table 1, compound Ia-A, compound of the formula Ia-B, compound of the formula Ia-C, compound of the formula Ia-E, compound of the formula Ia-F, compound of the formula Ia-H, compound of the formula Ia-I, compound of the formula Ia-G from Table 2 and combinations thereof.

15. A composition according to claim 12, wherein the at least one therapeutic agent may be an analgesic, a non-steroidal anti-inflammatory, cortisone, a cortisone derivative, a muscle relaxant or combinations thereof.
